SEQUENCE LISTING (8 sequences)

SEQ ID NO: 1 - protein, 22 aa, Artificial Sequence
  Synthetic peptide
GSCGCTVRHEGKCNCDTHIFVV

SEQ ID NO: 2 - protein, 23 aa, Artificial Sequence
  Synthetic peptide
ETCSGAGRKATAGCPCDTHILTM

SEQ ID NO: 3 - protein, 20 aa, Artificial Sequence
  Synthetic peptide
ADCLPEDVPACNCDSHTLVI

SEQ ID NO: 4 - protein, 21 aa, Artificial Sequence
  Synthetic peptide
GVCEHDERVPRCNCDTHVIVR

SEQ ID NO: 5 - protein, 20 aa, Artificial Sequence
  Synthetic peptide
GVCEPDDYYPCNCDTHTIVK

SEQ ID NO: 6 - protein, 23 aa, Artificial Sequence
  Synthetic peptide
GSCGGNCEFLDFPCTCDTHIIAR

SEQ ID NO: 7 - protein, 20 aa, Artificial Sequence
  Synthetic peptide
GDCQPDDYTPCNCDTHVLVK

SEQ ID NO: 8 - protein, 22 aa, Artificial Sequence
  Synthetic peptide
GECGCEKRPQPCNSCDVHVVTR

(19) United States
(12) Patent Application Publication
(10) Pub. No.: US 2018/0055913 A1
(43) Pub. Date: Mar. 1, 2018

(54) RECOMBINANT COLLAGEN IV SURROGATES AND USES THEREOF

(71) Applicant: VANDERBILT UNIVERSITY, Nashville, TN (US)

(72) Inventors: Billy G. HUDSON, Brentwood, TN (US); Christopher F. CUMMINGS, Brentwood, TN (US); Vadim PEDCHENKO, Nolensville, TN (US); Kyle BROWN, Nashville, TN (US); Roberto VANACORE, Nashville, TN (US)

(21) Appl. No.: 15/329,900

(22) PCT Filed: Jul. 29, 2015
(86) PCT No.: PCT/US15/42687

§ 371 Date: Oct. 13, 2017

Related U.S. Application Data
(60) Provisional application No. 62/030,170, filed on Jul. 29, 2014.

(51) Int. Cl. A61K 38/39

(52) U.S. Cl. CPC A61K 38/39

(57) ABSTRACT

The disclosure describes compositions that mimic certain structural and functional characteristics of collagen IV. Additionally provided are methods for the recombinant production of said compositions and particular methods of use.

This application claims benefit of priority to U.S. Provisional Application Ser. No. 62/030,170, filed Jul. 29, 2014, the entire contents of which are hereby incorporated by reference.

### STATEMENT REGARDING FEDERALLY-FUNDED SUPPORT

This invention was made with government support under grant numbers RO1 DK18381, DK18381-38S1 and 2PO1 DK065123 awarded by the National Institutes of Health. The government has certain rights in the invention.

### BACKGROUND OF THE INVENTION

### 1. Field of the Invention

The present invention relates generally to the fields of biology and medicine. In particular, the invention relates to collagen IV surrogates and uses thereof.

### 2. Description of Related Art

Collagen IV scaffolds are critical components of basement membranes (BM), a specialized form of extracellular matrix that underlies all epithelia in metazoa from sponge to human. Collagen IV molecules are assembled into networks that support the assemblage of BM components (Hudson et al., 2003). The scaffolds confer structural integrity to tissues, provide a foundation for the assembly of other macromolecular components, and serve as ligands for integrin cell-surface receptors that mediate cell adhesion, migration, growth and differentiation (Moser et al., 2009; Hynes, 2002; Yurchenco and Furthmayr, 1984). The networks also participate in signaling events in Drosophila development, in the clustering of receptors in the development of mammalian neuromuscular junction (Fox et al., 2007), and they are involved in autoimmune and genetic diseases (Gould et al., 2006; Gould et al., 2005; Hudson et al., 2003).

The collagen IV networks are assembled by oligomerization of triple-helical protomers by end-to-end associations and by intertwining of triple helices through their N- and C-terminal domains (Khoshnoodi et al., 2008; Khoshnoodi et al., 2006). At the C-terminus, two protomers associate through their trimeric non-collagenous (NC1) domains forming a hexamer structure. The protomer-protomer interface is covalently crosslinked, a key reinforcement that strengthens the structural integrity of networks. In the case of humans, the crosslink also confers immune privilege to the collagen IV antigen of Goodpasture autoimmune disease (Vanacore et al., 2008; Borza et al., 2005).

Structural integrity of the network has been shown to be important for the progression of several diverse medical conditions. Genetic loss of the α345 collagen IV network provides a molecular basis for Alport's disease, while mutation to the α112 collagen IV network can be a causal factor of vascular instability and stroke. Relatedly, while aortic aneurisms have an unknown etiology in humans, experimental models of aortic aneurisms are induced by destruction of the collagen IV network, suggesting that a population of human patients may similarly be in need of support for their collagen IV networks. Finally, several eye diseases have been clinically and/or experimentally associated with loss or damage to collagen IV or its associated proteins, including peroxidasin.

Damage to the collagen IV network may occur during normal ageing or as a result of chronic stressors. For example, advanced glycation end products may accumulate on collagen IV in diabetes, and thickening of the basement membrane is a hallmark seen in diabetic patients. In the eye, BM thickening within the retina is reported in aged eyes (Booji et al., Prog. Ret. Eye Res., 2010). Perturbation of the network has also been observed in many cancers.

Autoimmunity towards collagen IV is observed in Goodpasture's disease, being characterized by pathogenic autoantibodies that target the α345 collagen IV protomer in lungs and kidneys. Patients experience acute onset of severe symptoms, with medical treatment focused on reducing the circulating titer of autoantibodies.

Native collagen IV heterotrimeric molecules are known to spontaneously assemble into scaffold structures through complex intermolecular interactions. McCall et al teach that the formation of scaffolds is critical to at least some of the native functions of collagen IV in vivo (McCall et al., Cell, 2014). However, the technical challenges of manipulating these scaffolds have presented great hurdles towards harnessing any clinical utility of these proteins. Moreover, the complex folding requirements of collagen IV have foiled many previous efforts to efficiently produce recombinant versions of the heterotrimeric forms of the protein. Thus, while the clinical importance of collagen IV is being realized, the inventors suggest there is significant need for compositions and methods that effectively target and functionally modulate collagen IV in patients.

### SUMMARY OF THE INVENTION

Thus, in accordance with the present invention, there is described herein a composition of matter that (a) is a recombinant heterotrimeric protein complex that folds into conformations resembling native collagen IV heterotrimeric proteins; (b) contains NC1 and collagenous domains where the collagenous domain comprises one or more (Gly-Xaa-Yaa) triplet sequences; (c) self-assembles into its quaternary protein structure under the activity of the NC1 domains and below 37° C.; and (d) is recombinantly engineered to possess reduced antigenicity relative to native α345 collagen IV. Optionally, the protein may (a) be recombinantly engineered to possess a 7S domain at the N-terminus, affinity purification sequences or tags to assist in purification; or a fluorescent protein; (b) be recombinantly engineered and/or enzymatically processed to not contain the sequence (Gly-Pro-Hyp); (c) be recombinantly engineered to prevent the heterotrimeric NC1 termini from assembling into a larger hexameric complex between two adjacent heterotrimeric proteins; (d) possess amino acid sequences within the collagenous domain where two or more (Gly-Xaa-Yaa) triplets are separated by up to thirty (30) amino acids; (e) be conjugated to therapeutic compounds such as anti-angiogenesis or cancer chemotherapeutics; (f) be recombinantly engineered to contain a cysteine-rich region between the NC1 and collagenous domains, selected from SEQ NO 1 through SEQ NO 8; (f) possess binding sites for one or more of the following molecules: nidogen, usherin, fibronectin, laminin, chondroitin sulfate proteoglycan, heparin sulfate proteoglycan, factor IX, glycoprotein VI, heparin, heat shock protein 47, prolyl 3-hydroxylase, prolyl 4-hydroxylase, glycosyltransferase, goodpasture antigen binding protein, bone morphogenic protein 4, transforming growth factor β type 1, osteonectin, collagen VII, decorin; and/or (f) possess binding sites for one or more of the following cellular receptors: integrin α1β1, integrin α2β1, integrin α3β1, integrin αVβ3, integrin αVβ5, discoidin domain receptor 1, discoidin domain receptor 2, or cluster of differentiation 47 (CD47).

The composition may be used to treat the cause or symptoms of a disease in a patient when effectively administered to the patient. Furthermore, there are provided methods for manufacturing, packaging, and effectively administering said composition to a patient. The patient may be suffering from cancer, anterior eye disease, posterior eye disease, macular degeneration, glaucoma, fibrosis, Goodpasture's Disease, Alport's Syndrome, autoimmune disease, cardiovascular disease, aortic aneurism, stroke, chronic wound, surgical wound, connective tissue disease, skin disease, or any other disease or condition involving collagen IV.

The structure of the recombinant protein may be controlled with respect to the assembly of the heterotrimeric form and the ability of two heterotrimers to interact at their NC1 C-termini. The assembly of heterotrimers may be regulated via temperature, where the heterotrimer spontaneously assembles at temperatures below 37° C. yet is destabilized at higher temperatures. Such control may be advantageous for conjugating therapeutic compounds during recombinant protein synthesis. Once in its heterotrimeric form, the ability of the recombinant protein to interact with another similar protein via the C-terminal NC1 domains may be controlled by adjusting the concentration of chloride or bromide in the local chemical environment. The NC1 domains of adjoining recombinant proteins will associate when the local chloride or bromide concentrations are above 30 mM. Conversely, solutions of the recombinant protein may be prevented from forming NC1 hexamers by maintaining chloride or bromide concentrations below 30 mM. For example, the recombinant proteins may be induced to bind endogenous collagen IV scaffolds within a subject if, prior to administration, the recombinant proteins are stored in a formulation containing low amounts of chloride or bromide. In this situation, upon injection into the bloodstream of the patient, the recombinant proteins will become activated within their NC1 domains and will be able to interact with endogenous compatible collagen IV NC1 domains.

In one embodiment, the recombinant collagen IV may be administered to a patient for the purpose of recognizing and binding specific molecular targets, such as cell membrane integrins or antibodies, within a patient. The recombinant proteins may be genetically modified to remove arginine-76, asparaginine-187, glutamic acid-175, and/or arginine-179 (numbered beginning with the start of the NC1 domain) to prevent the formation of NC1 hexamers regardless of halide content within the buffer system. In this form, the recombinant protein may be useful for binding soluble molecules, antibodies, or cells. Considering that many cancers express collagen-binding integrins on their surface, including metastatic tumors, these recombinant collagen IV might be used to identify solid tumors and/or circulating cancer cells using standard imaging (MRI, immunofluorescence). Alternatively, they may be useful as a treatment for Goodpasture's patients by selectively binding pathogenic auto-antibodies that target collagen IV.

In another embodiment, recombinant collagen IV may be induced to join with an adjacent collagen IV protomer, of recombinant or natural origin, via the formation of an NC1 hexamer when in the presence of an appropriate concentration of halide, such as 100 mM chloride. This may be accomplished by introducing the recombinant collagen IV into a serum-based solution and providing a second available NC1 trimer for complimentary binding, where the second NC1 trimer is extracted from tissue, is recombinantly produced, or is a naturally-expressed protein in the patient undergoing treatment. The resultant NC1 hexamer may be further acted on by HOBr, such as through the activity of peroxidasin and a bromide salt, in order to form sulfilimine crosslinks within the hexamer. The recombinant collagen IV may be formulated with appropriate amounts of bromide salts or bromide-based compounds, for co-administration to the subject, in order to promote sulfilimine formation following administration.

In another embodiment, recombinant collagen IV may be induced to join with three other adjacent collagen IV protomers, of recombinant or natural origin, via the formation of 7S dodecamers at the N-termini of the protomers. This may be accomplished through the enzymatic activity of lysyl oxidase-like 2, which requires a copper ionic cofactor, and providing three available 7S heterotrimer for complimentary binding where the 7S domains are extracted from tissue, are recombinantly produced, or are naturally-expressed proteins in the subject undergoing treatment. Such an embodiment may require that the recombinant collagen IV be formulated with copper ions or copper-based compounds, for co-administration to the subject, in order to promote 7S crosslinking within the subject.

In another embodiment, the recombinant collagen IV may serve as a platform for the delivery of one or more therapeutic drug compounds to specific molecular targets within a patient. A diverse array of drugs may be conjugated via genetic engineering and/or chemical reaction(s) to this recombinant protomer platform including biologic-based compounds as well as small molecules. For example, a recombinant growth factor may be attached onto the recombinant collagen IV via molecular biology or through a biochemical binding event between the two recombinant products. Alternatively, the recombinant collagen IV may be genetically engineered to express one or more specific chemical targets, such as lysine residues, so that one or more small molecule drugs may be conjugated to the recombinant collagen IV via the appropriate chemical reaction(s). As one example, a specific recombinant collagen IV with conjugated anti-cancer drug may be injected into a cancer patient for the purpose of binding specific integrin targets on the tumor cells, and thereby deliver the drug compound to the tumor target. In another example, a recombinant collagen IV-growth factor complex may be therapeutically applied to a patient suffering from a chronic wound, where the collagen IV domains in said complex would be activated by biologic fluids to bind damaged collagen IV networks for the purpose of promoting wound closure and tissue regeneration.

In another embodiment, the recombinant collagen IV may be therapeutically administered to individuals with genetic diseases caused by mutations in collagen IV such as but not limited to Alport's Syndrome and thin basement membrane disease; transcription factors that are responsible for the tissue-specific expression of collagen IV; chaperone proteins or modifying enzymes that assist in the natural production of sulfilimine-crosslinked collagen IV scaffolds, such as but not limited to peroxidasin, lysyl hydroxylase, heat-shock protein 47, prolyl-3-hydroxylase, protein disulfide isomerase, prolyl-4-hydroxylase, and peptidyl prolyl cis-trans isomerase; or other proteins such as growth factors. In these cases, recombinant collagen IV may replace missing, mis-folded, or damaged collagen IV scaffolds or provide an immobilized surface that enhances the activity of mutated or otherwise damaged proteins.

In another embodiment, the recombinant collagen IV may be designed to express one, two, three, or more binding sites for cell surface receptors such as but not limited to integrins or discoid domain receptor 1; other extracellular matrix molecules such as but not limited to heparin sulfate proteoglycans, laminin, and fibronectin; or molecules such as but not limited to growth factors. The recombinant collagen IV may express multiple binding sites in order to immobilize two, three, or more targets via a single recombinant collagen IV protomer. For example, the recombinant product might be designed to bind two or more integrins in order to strengthen any downstream intracellular signaling that may result. Alternatively, the recombinant collagen IV may possess multiple yet different binding sites in order to immobilize a combination of cellular receptors and/or extracellular molecules in order to stimulate a sophisticated biological effect. For example, the recombinant collagen IV may possess binding sites for a specific integrin as well as a specific growth factor in order to function as a protein scaffold that facilitates growth factor-derived signal transduction events.

In another embodiment, sufficient quantities of the recombinant collagen IV may be produced for the purpose of assembling synthetic extracellular collagen IV scaffolds with bioactivity. These synthetic scaffolds may be designed to resemble the three-dimensional architecture, chemical composition, and mechanical properties of native, tissue-derived collagen IV scaffolds. These synthetic scaffolds may be acted on by enzymes such as peroxidasin and/or lysyl oxidase in order to form sulfilimine crosslinks and 7S crosslinking, respectively. Additional extracellular matrix proteins may be added to the scaffold in order to modify the structure and bioactivity, including but not limited to laminins, heparin sulfate proteoglycan, chondroitin sulfate proteoglycan, nidogen, fibronectin, and heparin. Further modifications to the scaffold may be made by attaching growth factors to bind the scaffold. These scaffolds, consisting of recombinant collagen IV either alone or in combination with other proteins, enzymes, molecules, may be used to therapeutically promote and guide tissue regeneration, facilitate the manufacturing of cultured organs for surgical transplantation, enable the advancement of cell culturing techniques, and catalyze biologic processes that require multiple enzymatic steps.

In another embodiment, the recombinant collagen IV may be genetically modified to prevent undesirable side effects upon administration to patients. For example, Pokidysheva et al. teach that the GlyProHyp sequence in collagen IV may bind platelet-specific glycoprotein VI (GPVI) (Pokidysheva et al., 2013), thus activating a pro-thrombotic pathway. The primary amino acid sequence of the recombinant collagen IV may be thus be genetically or enzymatically modified to prevent the occurrence of GlyProHyp as a means for mitigating the risk of triggering thrombosis via contact between the recombinant collagen IV protomer and blood products. The risk of side effects may also be mitigated by formulating the recombinant collagen IV with an anticoagulant such as heparin.

In yet another embodiment, a method for manufacturing the composition. The recombinant proteins may be individually expressed in mammalian cell culture, such as Chinese Hamster Ovary (CHO) cells, before being combined in the appropriate stoichiometry. Preferably, assembly of the heterotrimeric protomer will occur between 15 and 30° C., or at or near room temperature, and in a buffer containing preferably less than 1 mM halide ion, and including no halide ion. Upon assembly, the cysteine-knot may be allowed to spontaneously form or be catalyzed via chemical or enzymatic reaction.

In yet another embodiment, an alternative method for manufacturing the composition. The three desired chains may be recombinantly co-expressed in a single mammalian cell line, such as Chinese Hamster Ovary (CHO) cells. The desired heterotrimeric end product is secreted from the cells, in a properly folded conformation, and purified using standard biochemical techniques for manipulating collagen IV.

In yet another embodiment, a method of packaging the composition, and more specifically, using a solution containing halide concentration below 15 mM. Better yet, the solution may contain halide concentration below 1 mM. In this packaging, the composition will be activated to form collagen IV scaffolds by encountering a fluid with halide levels above 30 mM, or preferably above 50 mM, or ideally around 100 mM, such as the concentrations of chloride that are normally found in blood. Thus, the composition may be packaged and stored in an inactive state, while subsequently becoming activated to form a collagen IV scaffold upon being injected into a patient's bloodstream or other suitable routes of administration.

In another embodiment, the recombinant collagen IV may serve as a diagnostic platform for identifying patents who are at risk of collagen IV-associated diseases and/or disorders. Such diagnostic applications would involve conjugating one or more imaging agent(s) to the recombinant protomer using similar techniques as described above for the conjugation of drug compounds. When given to a patient, a diagnostic recombinant protomer may be designed to bind specific integrin targets or alternatively bind collagen IV targets. This may be useful in identifying areas where collagen IV scaffolding is in disrepair and may be a causative or contributing factor of disease, such as in predicting hemorrhagic stroke or monitoring cancer progression. Alternatively, a diagnostic recombinant protomer may be useful in assessing a wound caused by medical operation, traumatic wound, chronic wound, natural aging, exposure to environmental factor or disease. Such a diagnostic strategy for wounds might include labeling the damaged or nascent collagen IV scaffolding that is present in or around the wound bed, thereby either facilitating the degree of tissue damage within a wound or monitoring the healing process within a treated wound, respectively.

Considering that an individual recombinant protomer may bind a complementary protomer, of either recombinant or natural origin, upon activation by normal serum concentrations of chloride, it is also envisioned that the recombinant protomer platform may be used as a kit for bringing two or more compounds into relatively close proximity to each other. In this embodiment, at least one compound would be conjugated to one of the pairing protomers while the other compound is conjugated to the complimentary protomer. Upon activation by appropriate salt concentration, the recombinant protomers will be induced to bind together, thereby bringing the conjugated agents into their desired proximity. For medical applications, the recombinant protomers may be activated prior to administration to a subject or they may be activated in vivo after administration by the normal concentrations of chloride within the body fluids of the patient. Such a kit may also be utilized as an experimental reagent in certain biomedical research applications where two or more agents are desired to be in close proximity.

In another embodiment, inactivated recombinant protomers may be used as an antigen to generate antibodies that recognize the trimerized NC1 domains of collagen IV, and particularly against the surface area of trimers that is buried within the NC1 hexamer structure. Previous attempts to generate such an antibody have not been feasible due to lack of an antigen source that faithfully reproduces the three dimensional structure of native trimerized NC1 domains. This disclosure solves a long-standing research need of producing recombinant collagen IV NC1 trimers that are accurately folded. Antibodies that recognize collagen IV trimers may possess therapeutic utility by activating an immune response against tumor sites that generate large amounts of nascent collagen IV scaffolding.

In yet another embodiment, a method of disrupting the assembly of nascent collagen IV scaffolds in various disease states, such as in treating tumor angiogenesis. This method involves using an antibody or Fab that binds in the trimer-trimer interface of collagen IV NC1 hexamers, thus destabilizing the nascent collagen IV scaffold and resulting in the impairment of further tissue development at the site of disease.

In yet another embodiment, a method for in vivo labeling sites of collagen IV scaffold assembly or sites where the collagen IV scaffolds are perturbed, such as due to the loss of sulfilimine crosslinking. This method involved administering to the patient an antibody or Fab that (1) binds the trimer-trimer interface region of collagen IV hexamers and (2) is tagged with any commonly used molecular marker suitable for in vivo or clinical diagnostics.

The subject being treated may have incurred a medical operation, traumatic wound, chronic wound, natural aging, exposure to an environmental factor, or genetic disease. Bromide, chloride, and copper concentrations may be measured through mass spectroscopy, column chromatography, inductively coupled plasma mass spectrometry, neutron activation analysis, energy dispersive x-ray fluorescence, and particle induced x-ray emission. The subject may be a non-human animal or a human. Administering may comprise oral, intravenous, intra-arterial, subcutaneous, transdermal or topical administration, or systemic administration or administration to or local/regional to a site of healing.

It is contemplated that any embodiment discussed in this specification can be implemented with respect to any method or composition of the invention, and vice versa. Furthermore, compositions of the invention can be used to achieve methods of the invention.

The use of the word “a” or “an” in the claims and/or the specification may mean “one,” but it is also consistent with the meaning of “one or more,” “at least one,” and “one or more than one.”

Throughout this application, the terms “about” and “approximately” indicate that a value includes the inherent variation of error for the device, the method being employed to determine the value, or the variation that exists among the study subjects. In one non-limiting embodiment the terms are defined to be within 10%, preferably within 5%, more preferably within 1%, and most preferably within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The invention may be better understood by reference to one or more of these drawings in combination with the detailed description of specific embodiments presented herein.

FIGS. 1A-C: The NC1 domain is a primary junction point in collagen IV network assembly in basement membranes. (FIG. 1A) Basement membranes interact directly with most eukaryotic cell types enabling tissue functions. The basement membrane is a highly organized extracellular matrix where collagen IV networks function as scaffolds tethering ECM molecules and providing tensile strength. (FIG. 1B) During network assembly two triple-helical protomers self-associate at the NC1 domain, whereas four collagen IV protomers associate at the 7S domain. (FIG. 1C) Crystal structures reveal multiple ion binding sites along the NC1 inter-protomer interface.

FIGS. 2A-F: CT is required for NC1 Hexamer assembly. (FIG. 2A) Dissociation of purified bLBM hexamer (black line) into constituent NC1 monomers in Cl-free Tris-acetate buffer (red line). Representative SEC profile shown. (FIG. 2B) Reassembly of bLBM hexamer after incubation of concentrated NC1 monomers in the presence of 100 mM NaCl for 24 hr at 37° C. (FIG. 2C) Yield of reassembled bLBM hexamer is dependent on NaCl, while concentration of monomers decrease in proportion to hexamer formation. (FIG. 2D) Effect of monovalent anions tested as sodium salts at 100 mM on the assembly of bLBM hexamer from NC1 monomers. The physiologically relevant concentration of 100 μM NaBr did not support hexamer assembly. (FIG. 2E) K+ and Na+ yield similar amounts of hexamer. Cations tested at 100 mM of their Cl⁻ salt. (FIG. 2F) Ca²⁺ ions at 1 mM, does not support hexamer formation from LBM NC1 monomers in Cl-free environment (see FIG. 9G)

FIGS. 3A-F: Design, production, and characterization of recombinant protomers. (FIG. 3A) Model of Cl⁻ binding site, based on crystal structure of 112 NC1 hexamer. (FIG. 3B) Model of recombinant proteins with integrin α2α1 binding site engineered within triple helix region. Helix comprised 84 amino acid region from α1 and α2 chains immediately adjacent to NC1 domains (see FIGS. 10A-B). (FIG. 3C) Purified α1 and α2 recombinant monomers eluted as a single peaks at 14.5 ml by SEC column. (FIG. 3D) Product of recombinant protomers following in vitro assembly (see FIGS. 11A-E). Peaks identified as monomers (14.5 ml), protomers (P, 11 ml) and protomer dimers (P₂, 9 ml). (FIG. 3E) Functional integrity of protomer helices (P, P₂) shown α2 I-domain solid-phase binding assay. As expected, monomers (M) were inactive. Pretreatment of protomers or protomer dimers with bacterial collagenase abolished integrin-binding activity. Experiment performed in triplicate. Error bars represent ±1 SD. (FIG. 3F) Cell adhesion of HT-1080 cells is supported by recombinant protomers and protomer dimers, but not monomers, and abolished by collagenase pretreatment. Experiment performed in triplicate. Error bars represent ±1 SD.

FIGS. 4A-C: Protomer self-assemble while network self-assembly requires Cl⁻. (FIG. 4A) P2 dissociated into monomeric (M) chains through controlled steps. In TBS, the recombinant proteins existed as P2 (black line), yet dissociated into P in TrisAc buffer (red line), and dissociated into M upon heating at 37° C. (blue line). (FIG. 4B) Controlled reassembly of monomers into protomers (P). M samples (blue line) spontaneously assembled into P in TrisAc after 24 hr at 20° C. (red line), notably occurring without Cl⁻. Incubation in 100 mM Cl− yielded P2 (black line) as the reassembled protomer dimer. (see FIG. 12, Table S1). (FIG. 4C) Protomer dimers crosslinked by PXDN (P2X) are completely resistant to dissociation in Cl-free environment (left), while un-crosslinked dimers (P2) dissociate into protomers (P, right). Inset shows SDS-PAGE of P2 and P2X samples, demonstrating crosslinking in P2X only (see FIGS. 13A-C).

FIGS. 5A-E: Cl⁻ triggers a molecular switch that controls protomer assembly into higher order networks. (FIG. 5A) In the absence of Cl⁻, R76 can form an intramolecular salt-bridge with D78 and/or E40. (FIG. 5B) Extracellular Cl⁻ disrupts the R76-D78 salt-bridge via electrostatic screening. Hydrogen bonds occupancies decrease in the presence of Cl⁻. (FIG. 5C) Specific binding activity of Cl⁻. The ion binds directly within a nested region where Cl⁻ coordinates with the backbone amides or R76 and D78, limiting their ability to reform an intramolecular salt-bridge (see also FIG. 14). (FIG. 5D) R76 bridges the protomer interface to form an intermolecular salt-bridge with E175 and an end-on coordination with N187. Moreover, R179 may interact with Cl⁻ directly, lending further stability to the interface. (FIG. 5E) R76A recombinant mutants to assemble protomers, but not protomer dimers (P2; see FIGS. 15A-D), confirming essential importance of the switch during assembly.

FIG. 6: Key residues of Cl-mediated assembly switch are defining features of collagen IV. In all organisms examined through Placozoa, the essential R76 and D78 residues are present in at least one collagen chain while direct electrostatic interaction with Cl⁻ is possible in all organisms represented (see FIGS. 16 and 17; Table S2). The presence of N187 determines whether a regular or networked salt-bridge is capable of forming. Ca²⁺-mediated electrostatic interactions are limited to Deuterostoma. Table on right enumerates the salt-bridges and electrostatic interactions per hexamer, as found at the trimer-trimer interface.

FIGS. 7A-C: Multi-functional NC1 domains control Collagen IV protomer and network assembly. (FIG. 7A) Collagen IV NC1 domains nucleate protomer assembly by controlling monomer stoichiometry, specificity, chain register, and preventing aggregate-induced ER stress intracellularly. (FIG. 7B) The elevated extracellular chloride concentration prompts protomers to form NC1 hexmers. The assembly is covalently reinforced by sulfilimine crosslinks, as formed by peroxidasen (PXDN). (FIG. 7C) Highly organized collagen IV scaffolds form the backbone of basement membranes.

FIGS. 8A-K: Chloride is required for hexamer assembly (related to FIGS. 2A-F). Size-exclusion chromatography (SEC) elution profiles of native LBM NC1 hexamer in TBS (FIG. 8A) and LBM hexamer after dissociation in 6M guanidine-HCl (FIG. 8B) or 8M urea (FIG. 8C). Dissociation results in the disappearance of hexamer peak at 14 ml and formation of NC1 monomers peak at 16.3 ml. (FIG. 8D) Dissociation of uncrosslinked NC1 hexamer from PFHR9 cells after dialysis in Tris-acetate buffer (red line). SEC profile of the hexamer in TBS is shown as a control (black line). (FIG. 8E) Phosphate buffer induces dissociation of uncrosslinked NC1 hexamers from PHFR9 cells. Dialysis in phosphate buffer (10 mM, pH 7.4) results in dissociation of hexamers deposited by cells grown in the presence of KI (red line) or phloroglucinol (blue line) to inhibit crosslinking. Same hexamers are stable in PBS as indicated by a single peak eluted at 14 ml (black line). (FIG. 8F) Dissociation of LBM hexamers after dialysis in phosphate buffer (10 mM, pH 7.4). (FIG. 8G) Composition of the hexamer reassembled from LBM NC1 monomers in the presence of Cl⁻. NC1 monomers purified upon dissociation of LBM hexamer in Tris-acetate buffer were concentrated, and incubated with 100 mM NaCl for 24 hrs at 37° C. After separation by SEC, subunit composition of reassembled hexamer has been analyzed by Western blotting using monoclonal antibodies to α1NC1 and α2NC1 domains, respectively. Lanes: 1, LBM hexamer; 2, purified LBM NC1 monomers; 3, reassembled NC1 hexamer. Positions of NC1 monomers and sulfilimine crosslinked dimers are indicated on the right. (FIGS. 8H-J) Characterization of the hexamer assembly reaction. Effects of incubation temperature (FIG. 8H), starting concentration of NC1 monomers (FIG. 8I), and incubation time (FIG. 8J) on the yield of reassembled LBM hexamer were examined in the presence of 150 mM chloride. The assembly reaction reached equilibrium by 24 hours (FIG. 8J). Assembly quantified from SEC elution profiles following reaction as a percentage of the hexamer peak from the total peak area. (FIG. 8K) Chloride induces hexamer assembly from dissociated PFHR9 NC1 monomers. After dissociation in Tris-acetate buffer, one part of the sample containing NC1 monomers was directly separated by SEC (black line), while the second part was separated after pre-incubation with 100 mM Cl⁻ (red line) resulting in the formation of hexamer concomitant with the loss of NC1 monomers. No changes were observed in 7S peak, which served as internal control.

FIGS. 9A-G: Calcium and potassium ions are not required for hexamer assembly (related to FIGS. 2A-F). (FIG. 9A) Molecular modeling of K⁺ ions within the hexamer complex. (FIG. 9B) Effect of monovalent cations on LBM hexamer assembly. All cations were tested in chloride form at 100 mM and induced the formation of the comparable amounts of hexamer. In contrast, switching of chloride to acetate exemplified with cesium salts resulted in complete loss of hexamer formation, indicating strong chloride dependence of assembly. (FIG. 9C) Molecular model of Ca²⁺ within a divalent cation binding site formed by E¹⁴⁹ and D¹⁴⁸. Ca²⁺ binding is seen only in the α2 monomers. (FIG. 9D) Distances between individual calcium ions and the carboxyl carbon of aspartic acid residues were monitored during MD simulations with respect to solvent Cl⁻ concentration. (FIG. 9E) In a physiologically relevant concentration range (0.1-10 mM) CaCl₂ alone does not induce hexamer assembly. Under the same conditions, chloride (100 mM NaCl) induced formation of LBM hexamer from NC1 monomers. (FIG. 9F) Complexing of residual Ca²⁺ with EDTA (red line) has no effect on hexamer assembly compared with TBS buffer alone (black line). (FIG. 9G) Calcium ions may potentiate hexamer formation in the presence of chloride. In the presence of additional CaCl₂ at physiological concentration (1 mM, red line) more hexamer formed from LBM NC1 monomers compared to the 100 mM NaCl alone (black line). SEC profiles are displayed.

FIGS. 10A-B: Design and Expression of Recombinant Protomer (rProt) (related to FIGS. 3A-F). (FIG. 10A) Schematic of recombinant protomers (rProt), following heterotrimeric assembly. The rProt contains a single, site-specific integrin α2β1 binding site and N-terminal FLAG tag for affinity purification (N-terminus shown on left). (FIG. 10B) Primary amino acid with substitutions introduced into the α1 and α2 recombinant proteins to introduce the α2β1 integrin binding site displayed in red.

FIGS. 11A-H: Characterization of recombinant protomers (related to FIG. 3). (FIGS. 11A-C) Following collagenase digestion, P₂ (FIG. 11A), P (FIG. 11B), and M (FIG. 11C) peaks were compared to LBM (dashed chromatogram) by SEC. The digest converted P₂ into a hexamer-like peak, while P and M were converted into NC1 monomer-like peaks. (FIG. 11D) Western blot analysis of each SEC peak and its collagenase digest product were stained for α1 and α2 NC1 domains. Unfractionated samples (input) from both recombinant products served as controls. (FIG. 11E) ELISA analyses of fractions from recombinant hexamer peak using chain-specific antibodies indicate a 2:1 α1:α2 chain stoichiometry. LBM was used as a control. (FIG. 11F) SDS-PAGE electrophoresis of SEC peaks denatures each peak to monomers components at 35 kD. Resistance to proteolysis by trypsin and chymotrypsin was observed for peak 1 containing CB3 mini-protomer. Soybean trypsin inhibitor (SBTI) was used to quench digestion. (FIGS. 11G-H) The helical content of SEC peaks was measured by CD spectroscopy (FIG. 11G). Peak 1 containing CB3 mini-protomer had the highest helical content as observed by strong negative ellipticity at 198 nm and positive ellipticity at 220-235 nm. The thermal stability of CB3 mini-protomer was measured by CD and found to have two transition points at 30° C. and 66° C., corresponding to the melting temperatures of helices and NC1 domains, respectively (FIG. 11H).

FIGS. 12A-D: Electrostatic topology of NC1 subdomains (related to FIG. 4). (FIG. 12A) Protomer specificity is dictated by interactions of the VR3 and b-hairpin regions. Electrostatic surface potentials are rendered onto the NC1 monomer van der Waals surface revealing the VR3 and b-hairpin regions are predominantly charge neutral. (FIG. 12B) The trimer electrostatic surface potential reveals distinct pockets of charge on the trimer exterior. Specifically, the trimer-trimer interface is dominated by electro negative potential in the center cavity that surrounds the calcium binding site. In addition R76, G175, and R179 comprise discrete charge pockets (units=Boltzman's constant (k)×temperature (298 K)/electron charge (q)). (FIG. 12C) These pockets complement each other in trimer-trimer association. (FIG. 12D) The contribution of salt to the electrostatic interactions of monomer-monomer and trimer-trimer association were estimated using a non-linear Poisson-Boltzmann calculation. Salt has a favorable impact on a1A-a1B, a2C-a1A, and trimer-trimer association and a negative effect on a1B-a2C association.

FIGS. 13A-C: Sulfilimine Bonds Reinforce assembled hexamers (related to FIGS. 4A-C). (FIG. 13A) Peroxidasin catalyzes formation of sulfilimine crosslink in LBM hexamer which confers hexamer to resist dissociation in Tris-acetate. LBM monomers were reassembled into hexamer by incubating in TBS. Reassembled LBM hexamer was preincubated with PXDN, Br⁻, and H₂O₂. Inset displays SDS-PAGE of reassembled LBM hexamer prior (lane 1) and after PXDN treatment (lane 2), and native LBM hexamer (lane 3) to show positions of NC1 monomers and crosslinked dimers. (FIG. 13B) Hexamer assembly is a prerequisite for sulfilimine bond formation. Reassembled hexamers or dissociated NC1 monomers from PFHR9 cells were treated with HOBr (50 μM). After 5 minutes at 37° C. reaction was quenched with methionine. SDS-PAGE shows that crosslinking (dimer formation) occurred only with the hexamer as a substrate. FIG. 13 (C) Crosslinking with HOBr stabilizes LBM hexamer against dissociation in guanidine-HCl. Hexamer was reassembled from NC1 monomers in the presence of chloride, isolated by SEC and crosslinked with HOBr. After crosslinking protein was treated with 6 M guanidine-HCl for 30 minutes at 65° C. before SEC analysis (red line). In contrast, untreated LBM hexamer completely dissociated into NC1 monomers under the same conditions (black line). Inset displays SDS-PAGE of reaction input material (lane 1), LBM hexamer after HOBr treatment (lane 2), and native LBM hexamer (lane 3).

FIGS. 14A-D: Comparison b-hairpin atomic fluctuations (related to FIGS. 5A-E). (FIGS. 14A-D) Atomic fluctuations of the a1 monomer (FIG. 14A), α2 monomer (FIG. 14B), a112 trimer (FIG. 14C), and a112 hexamer (FIG. 14D) were measured in 0 (black) and 150 mM Cl⁻ (red). The b-hairpin and VR3 regions are highlighted by grey filled boxes. For α112 trimer and hexamer system the a1 A chain is depicted. Atomic fluctuations are projected onto representative structures (right panels)

FIGS. 15A-D: Assembly of R76A chimeras (related to FIGS. 5A-E). (FIGS. 15A-B) Recombinant R76A chimeras of both α1-CB3 (FIG. 15A) and a2-CB3 (FIG. 15B) constructs were expressed and analyzed by Western blot. (FIG. 15C) Assembly of R76A-α1-CB3 and R76A-a2-CB3 constructs produce a (1,1,2)-CB3 trimer, but not hexamer. (FIG. 15D) Heat dissociates the trimeric complex to monomeric components.

FIG. 16: Cystine Rich Regions from Ctenophore Collagen IV, for Inclusion in the Recombinant Protomers. Amino acid sequences of Ctenophore collagen IV, exemplifying cysteine rich region. Sequences obtained via RNAseq techniques. Cystines are in bold and enlarged. Red highlighted denotes start of NC1 sequence. A cysteine doublet is invariably located eight residues from the NC1. Cystine doublets typically found as CxC, where x is often N. These sequences may be inserted, in whole or in part, into the Protomers disclosed herein.

FIG. 17: Amino Acid Sequences for Cystine Rich Regions Used in the Claimed Invention.

FIG. 18: Proposed mechanism for inhibition of collagen IV assembly by antibodies.

FIGS. 19A-B: (FIG. 19A) SDS-PAGE of purified H22 IgG and Fab fragments. (FIG. 19B) Binding of purified H22 IgG and its Fab fragments to immobilized α2 NC1 domain by ELISA. The absorbance was measured after incubation with alkaline phosphatase-conjugated secondary anti-rat antibodies in the presence of substrate and quantified using a microplate reader.

FIGS. 20A-B: Gel-filtration FPLC profiles of monomeric α2 NC1 domain after incubation with H22 IgG (FIG. 20A) or Fab (FIG. 20B). The appearance of a new peak at 11.0 ml (in FIG. 20A) indicates the formation of an IgG:α2 complex. Small peaks at 12.4 ml and 16.3 ml represent free antibodies and α2 NC1, respectively. The appearance of a distinct new peak at 14.0 ml (in FIG. 20B) indicates the formation of the Fab:α2 complex. Small amounts of free α2 NC1 and H22 Fab formed a broad peak at 16 mL due to similar molecular weight.

FIGS. 21A-D: NC1 hexamers from bovine placental (bPBM) and lens (bLBM) basement membranes were of particular interest to this experiment due to a variable number of crosslinks (bLBM has significantly less crosslinks compared to bPBM). After incubation of bLBM (FIG. 21A) or bPBM (FIG. 21B) with H22 full-length IgG, resulting peaks remained at the positions of corresponding control peaks (12.4 ml and 13.6 ml), indicating the absence of the interaction. Similarly, the incubations of Fab fragments with bLBM (FIG. 21C) and bPBM (FIG. 21D) hexamers were consistent with the results from the full length IgG showing the absence of interaction.

FIGS. 22A-C: (FIG. 22A) In the control reaction, incubation of bLBM NC1 monomers in TBS resulted in efficient reassembly of NC1 hexamer (peak at 13.8 ml). (FIG. 22B) When H22 IgG were added, efficiency of hexamer reassembly was decreased (blue arrow) concomitant with the appearance of a new broadened peak at 12.0 ml representing α2 NC1:IgG complexes. (FIG. 22C) The addition of H22 Fab also inhibited hexamer formation as indicated by the reduced peak at 13.8 ml (blue arrow), as well as the appearance of a new peak at 14.4 mL indicating α2 NC1:Fab complex.

### DETAILED DESCRIPTION OF ILLUSTRATIVE EMBODIMENTS

Biologic matrices are essential and decisive factors in tissue development and function. The function of these extracellular surfaces is dependent on their biologic composition, structural organization, and stabilization via chemical crosslinks. Recent discoveries described below allow the control of these matrix characteristics, affecting a range of physiological processes including cellular proliferation and differentiation, tissue growth, vascularization, and disease pathology.

A key structural requirement of these matrices is an embedded collagen IV network that provides critical stability to the matrix (Poschl et al., 2004; Gupta et al., 1997; Borchiellini et al., 1996.). The establishment of these networks hinges on the activity of peroxidasin (PXDN), an enzyme that is embedded within matrices and crosslinks the C-termini of collagen IV heterotrimeric protomers. Recent discoveries now allow this enzyme to be functionally inhibited or activated through pharmacologic agents, enabling the fine-tuned control of collagen IV network assembly for the purpose of engineering biologic matrices with specific functional properties.

PXDN is a heme peroxidase that has been recently discovered to promote network assembly by forming sulfilimine bonds between the C-termini of adjoining collagen IV protomers. This catalytic activity is inhibited by pharmacologic treatment with either iodide or thiocyanate ions or with small molecules such as phloroglucinol or methimazole. The enzyme is upregulated during tissue growth, and also guides axon regrowth following neurologic injury (Gotenstein et al., 2010). Its cofactor requirements during sulfilimine bond formation include ionic bromide and an oxidizing source such as peroxide or molecular oxygen in combination with an electron-accepting compound such as flavin adenine dinucleotide. Enzymatic activity can be synthetically enhanced through the administration of one or more of these cofactors. A potential use for these cofactors may be to stimulate PXDN activity to promote wound healing, tissue regeneration, and neurologic growth due to injury or developmental defect. Additionally, stimulating PXDN activity via these cofactors may be used to prevent tissue degeneration due to disease, aging, medical treatment, medical operation, or environmental exposure.

The inventors have delineated the molecular mechanism of bond formation. They showed that PXDN catalyzes sulfilimine bonds directly within basement membranes using hypohalous acid intermediates. These findings provided the first known function for PXDN and highlight a biosynthetic role for conventionally toxic hypohalous oxidants. In addition, a key role for bromide in this reaction was established, providing a previously unknown connection between this chemical entity and tissue stability and repair.

Here, the inventors provide a distinct approach to increasing collagen IV structures. They have designed a variety of collagen IV surrogates for recombinant production, which can be used to substitute for collagen IV structures in vivo. They can also be used in the production of anti-collagen IV antibodies, previously unattainable due to correctly configured antigenic material.

### A. COLLAGEN IV, HUMAN PEROXIDASIN AND SULFILIMINE CROSSLINKS

1. Basement Membranes

In epithelial tissues, the cellular microenvironment is shaped through an organized milieu of signaling molecules, nutrient supply, cell-cell contacts, and mechanical parameters. Basement membranes (BMs) are defining features of this microenvironment, comprising specialized extracellular matrices that underlie epithelial cells and critically influence basic processes such as tissue morphogenesis and maintenance; organogenesis; nutrient diffusion; and cell polarity, differentiation, and migration (Daley and Yamada, 2013; Yurchenko, 2011; Pastor-Pareja and Xu, 2011). Consequently, alterations in the ultrastructure and composition of BMs occur alongside cancer progression and degenerative diseases such as macular degeneration (Lochter and Bissell, 1995; Ghajar et al., 2012; Booji et al., 2010).

Despite the key role of BM in influencing tissue behavior and health, it is challenging to obtain clinically meaningful information about the status of BM in a patient without performing an invasive biopsy. Certain techniques such as second generation harmonic imaging have recently emerged, although it is uncertain whether they have sufficient resolution to distinguish between healthy and perturbed BMs. The present invention provides compositions and methods that may enable a higher quality diagnostic tool for clinical and research use.

2. Collagen IV

Collagen IV (ColIV or Col4) is a type of collagen found primarily in the basal lamina. The collagen IV C4 domain at the C-terminus is not removed in post-translational processing, and the fibers link head-to-head, rather than in parallel. Also, collagen IV lacks the regular glycine in every third residue necessary for the tight, collagen helix. This makes the overall arrangement more sloppy and with kinks. These two features cause the collagen to form in a sheet, the form of the basal lamina. Collagen IV is the more common usage, as opposed to the older terminology of type-IV collagen. There are six human genes associated with it: COL4A1, COL4A2, COL4A3, COL4A4, COL4A5, COL4A6.

The alpha-3 subunit (COL4A3) of collagen IV is thought to be the antigen implicated in Goodpasture's Disease, wherein the immune system attacks the basement membranes of the glomeruli and the alveoli upon the antigenic site on the alpha-3 subunit becomes unsequestered due to environmental exposures. Goodpasture's Disease presents with nephritic syndrome, and hemoptysis. Microscopic evaluation of biopsied renal tissue will reveal linear deposits of Immunoglobulin G by immunofluorescence. This is classically in young adult males.

Mutations to the genes coding for collagen IV lead to Alport syndrome. This will cause thinning and splitting of the glomerular basement membrane. It will present as isolated hematuria, sensorineural hearing loss, and ocular disturbances and is passed on genetically in an X-linked manner.

3. Collagen IV Scaffolds

Collagen IV scaffolds are key components of basement membranes (BM), where they are critically influence BM morphology and function from an embedded location within the BM (Poschl et. al., 2004; Pastor-Pareja & Xu, 2011; McCall et al. 2014). These scaffolds perform an assortment of mechanical and signaling functions by tethering laminins, growth factors and other BM components into an organized bioactive matrix (Khoshnoodi, Pedchenko, and Hudson, 2008; Wang et al. 2008). The scaffolds confer structural integrity to tissues, provide a foundation for the assembly of other macromolecular components, and serve as ligands for integrin cell-surface receptors that mediate cell adhesion, migration, growth and differentiation (Moser et al., 2009; Hynes, 2002; Yurchenco and Furthmayr, 1984). Moreover, the scaffold itself is a ligand for cellular receptors such as integrins and discoidin domain receptor 1 (DDR1) (Parkin et al., 2011; Fu et al., 2013). The networks also participate in signaling events during the development and maintenance of tissues and organs, including epithelial, endothelial, vascular, renal, and neural tissues (McCall et al., Cell, 2014; Gould et al., 2005; Poschl et al., Development, 2004; Fox et al., 2007; Hudson et al., N. Engl. J. Med., 2003), and they are involved in autoimmune and genetic diseases (Kuo, Labelle-Dumais, and Gould, Hum. Mol. Genet., 2012; Gould et al., 2006; Gould et al., 2005; Hudson et al., 2003). Indeed, the ubiquitous and joint conservation of collagen IV and tissues throughout the Animal Kingdom implicate collagen IV scaffolds as a foundational requirement for tissue organization in animals (Fidler et al., 2014).

a. Structure

Collagen IV scaffolds are composed of heterotrimeric collagen IV protomers. These protomers are defined by an N-terminal 7S domain, a collagenous domain, and a C-terminal NC1 domain. 7S and collagenous domains adopt a helical structure, as is commonly seen in all collagen proteins, while NC1 domains are globular in structure. Protomers themselves contain three a chains. Humans possess six genetically distinct α chains, termed α1-6, yet collagen IV protomers in vivo are only seen in three distinct combinations (α112, α345, and α556). All α chains display similar domain structure as protomers (N-terminal 7S, collagenous domain, and C-terminal NC1 domains). Protomer assembly is initiated by self-assembly of the C-terminal NC1 domains, and is followed by helical winding in an N-terminal direction.

Collagen IV scaffolds display highly ordered junctions between and among protomers, suggesting that proper assembly is important for functional activity. The C-terminal NC1 domains of adjoining protomers assemble into NC1 hexamers, comprising six chains from two heterotrimeric protomers, for which x-ray structures are available (Sundaramoorthy et al., 2002; Vanacore et al., 2004). Electron micrographs of BMs also reveal 7S complexes, comprising N-termini from four protomers in a crosslinked structure, as well as lateral associations that form via intertwining helical collagenous domains (Yurchenko and Furthmayr, 1984). Moreover, protomers themselves are exclusively found in only three combinations of α chains (α112, α345, and α556).

b. Biologic Function

Collagen IV scaffolds are essential for the development, maintenance, and regeneration of tissues (Vracko, 1974; Gupta et al., 1997; Poschl et. al., 2004; Daley and Yamada, 2013; Yurchenko, 2011; Pastor-Pareja and Xu, 2011; Song and Ott, 2011; McCall et al. 2014). They are found within basement membranes underlying all epithelial and endothelial tissues. Consequently, pathologic disruption of collagen IV scaffolds can impact virtually any organ. Conversely, collagen IV scaffolds may serve as therapeutic targets for a wide variety of diseases and conditions. Moreover, these scaffolds may provide a key extracellular platform for tissue regeneration.

Collagen IV heterotrimeric protomers bind a diverse assortment of cellular and extracellular partners. Scaffolds promote interactions between cells and BMs, engage the interstitial matrix through collagen VII and anchoring fibrils, establish immobilized growth factor gradients, mechanically support overlying tissues, and provide a reservoir of signaling molecules (Wang et al., 2008; Parkin et al., 2011 and Fu et al., 2013).

Collagen IV protomers are found with three different combinations of α chains: α112, α345, and α556. In tissues, the α112 protomers are expressed throughout life while the other two protomers begin to be expressed after childhood. The α112 protomers interact with either other α112 protomers or α556 protomers, while the α345 protomers interact with themselves to form α345 networks. These protomers display distinct expression patterns in tissues, and likely serve separate biologic functions. The protomers contain numerous glycosylsations, hydroxylations, disulfide bonds, and binding sites for other proteins, glycoproteins, and cell receptors to bind. Known binding partners of collagen IV include nidogen, usherin, fibronectin, laminin, chondroitin sulfate proteoglycan, heparin sulfate proteoglycan, factor IX, glycoprotein VI, heparin, heat shock protein 47, prolyl 3-hydroxylase, prolyl 4-hydroxylase, glycosyltransferase, Goodpasture antigen binding protein, bone morphogenic protein 4, transforming growth factor 3 type 1, osteonectin, collagen VII, and decorin. In tissues, protomers assemble into crosslinked scaffolds that tether these binding partners within the extracellular matrix, specifically the basement membrane, which effectively modulates the overall function of these matrices.

c. Assembly

Collagen IV protomers assemble into collagen IV scaffolds through specific governing mechanisms, involving unique enzyme and chemical participants. The assembly of collagen IV scaffolds has emerged as a critical step in tissue morphogenesis, involving a combination of self-driven and enzymatically-catalyzed processes. C-terminal NC1 domains nucleate the self-assembly of heterotrimeric collagen IV protomers, simultaneously establishing chain register and selectively governing chain composition (six genetically-distinct α chains, α1-6) (Yurchenko and Furthmayr, 1984; Dolz, Engel, and Kuhn, 1988; Boutaud et al., 2000; Sundaramoorthy et al., 2002; Khoshnoodi et al., 2006). Within the BM, adjacent protomers interact through their heterotrimeric NC1 domains to form an NC1 hexamer (Khoshnoodi, Pedchenko, and Hudson, 2008). Tissue-derived NC1 hexamers possess novel sulfilimine crosslinks which form through the activity of peroxidasin (PXDN) and Br⁻ cofactor, while the catalytic mechanism harnesses hypobromous acid (HOBr) as an oxidizing reaction intermediate (Vanacore et al., 2009; McCall et al., 2014; Bhave et al., 2012). Perturbation of either PXDN or Br⁻ disrupts tissue architecture in Drosophila and leads to early lethality (McCall et al., 2014; Bhave et al., 2012). Beyond the NC1 domain, the collagenous domains of collagen IV self-associate, forming lateral interactions, while the 7S domains from for adjoining protomer assemble into a crosslinked structure.

i. Sulfilimine Crosslinks

The sulfilimine crosslinks are unique to collagen IV scaffolds, being unknown elsewhere in biology. Their presence is critical to sufficiently stabilizing the scaffold so as to support the diverse biologic functions of collagen IV.

Using mass spectrometry (MS) analyses of crosslinked tryptic (Tp) peptides and a smaller crosslinked post-proline endopeptidase (PPE) peptides, both derived from the α1α2α1 collagen IV network of placenta, it was found that Lys211 is modified to hydroxylysine (Hyl211) and that Hyl211 is covalently linked to Met93 forming a sulfilimine crosslink (Vanacore et al. 2009). In the α3α4α5 network, it was found that the sulfilimine crosslink connects the α3 and α5 NC1 domains, but the α4 NC1 domains are crosslinked at Lys211 instead of Hyl211, indicating that this post-translational hydroxylation modification is not a requirement for crosslink formation. Up to 6 sulfilimine bonds fasten the interface of the trimeric NC1 domains of two adjoining protomers, reinforcing the quaternary structure of the networks. Furthermore, the sulfilimine bond also occurs in the α3α4α5 collagen IV network because fragmentation pattern of its crosslinked tryptic peptides (Vanacore et al., 2008) is identical to that of the α1α2α1 network described herein. This sulfilimine linkage between Met and Lys/Hyl may not occur only in collagen IV but in other proteins as well.

Sulfilimine crosslinks are vital to the mechanical properties and function of basement membranes, due to their role in stabilizing collagen IV scaffolds. These crosslinks are the sole type of covalent crosslink at the C-terminal NC1 junctions in collagen IV. Animal models have revealed some of the effects of biochemically disrupting the structural integrity of collagen IV scaffolds. Inhibition of sulfilimine crosslink formation leads to collagen IV scaffolds that are thickened and split, disturbed tissue architecture, and embryonic or early development lethality (Bhave et al., 2012; McCall et al. and Cell, 2014).

ii. Peroxidasin

Peroxidasin (PXDN) is a heme peroxidase enzyme found within basement membranes. The enzyme forms sulfilimine crosslinks, acting on collagen IV in the extracellular space where it oxidized ionic Br− into hypobromous acid (HOBr) which subsequently serves as the oxidizing intermediate of the crosslinking reaction. In addition to Br−, the enzyme requires a second cofactor comprising an oxidizing source such as peroxide or molecular oxygen in combination with an electron-accepting compound such as flavin adenine dinucleotide.

Similar to the phenotype caused by loss of sulfilimine crosslinks in vivo, perturbation of PXDN via genetic mutation or pharmacologic inhibition yields abnormal tissue architectural phenotypes in zebrafish, nematodes, Drosophila, and humans (Fidler et al., 2014; Gotenstein et al., 2010; Bhave et al., 2012; McCall et al., Cell, 2014; Khan et al., 2011). Clinical cases are known of individuals with PXDN mutations, likely involving loss-of-function mutations, yielding a phenotype of disrupted tissue architecture in the anterior eye chamber causing juvenile cataracts (Khan et al., Am. J. Hum. Genet., 2011). Since PXDN requires Br− ions to form sulfilimine bonds, depletion of Br− can also disrupt tissue architecture, being confirmed in Drosophila as well as goats (McCall et al., Cell, 2014; Haenlein and Anke, Small Rumin. Res., 2011).

The accession nos. for human peroxidasin precursor protein and mRNA are NP_036425.1 and NM_012293.1, respectively, which are hereby incorporated by reference.

iii. Hypobromous Acid

Bromide ions are required for collagen IV sulfilimine bond formation, being oxidized by PXDN into HOBr which is the oxidizing intermediate of the crosslinking reaction. Due to this activity, Br⁻ occupies a critical function in the stabilization of tissue architecture. This function is necessary for animal life and represents the first essential function for the bromide ion in mammalian biology. The magnitude of this finding is only truly appreciated by independently considering the requirement for this specific halogen as well as the biosynthetic activity of the oxidant. On the one hand, the element bromine has lacked any essential function within animals prior to this discovered sulfilmine activity, with resulting ambiguity regarding its role in biology. Furthermore, its biologic relevance is often overshadowed by the significantly greater serum chloride concentration and the chemical reactivity of thiocyanate. On the other hand, hypohalous acids are commonly described for their capacity as destructive oxidants; useful within the immunologic toolkit but pathologic when unregulated as seen in atherosclerosis and other diseases associated with oxidative stress. The anabolic activity of HOBr during sulfilimine catalysis is partially analogous to the activity of oxidized iodide during thyroid hormone synthesis. Yet structural analysis of the products reveals an iodinated hormone that contrasts with the non-halogenated sulfilimine bond, strongly suggesting the utilization of distinct chemistry. In sufilimine bond formation, Br⁻ acts as a chemical catalyst and hypobromous acid the reactive intermediate.

iv. Crosslinked 7S Domains

The N-termini of collagen IV protomers are covalently assembled into 7S dodecameric domains through the enzymatic activity of LOX2, forming lysyl-lysine crosslinks within the dodecamer, and are further stabilized by additional covalent crosslinks. 7S dodecamer crosslinking may be prevented via the LOXL2 inhibitor β-aminopropionitrile (BAPN) or reinforced through the application of a LOX2 cofactor such as copper. LOXL2 forms aldehyde functional groups on target lysine residues, which then react to form the lysyl-lysine crosslinks via spontaneous chemical events.

7S domains provide critical rigidity to collagen IV networks and thereby impact the functioning of biologic matrices. The absence of crosslinks from these domains can prevent vascularization via destabilization of blood vessel basement membranes (Bignon, M, et. al. Blood, 2011). Targeting the 7S domain may be an effective strategy for blocking tumor angiogenesis. Further, collagen IV is a required element for some forms of liver metastasis (Burnier, J V, et. al. Oncogene, 2011). Therefore, pharmacologic modulation of 7S domains, via either the inhibition of LOXL2 crosslinking activity or the chemical cleavage of internal crosslinks, may be a potential therapeutic strategy for preventing tumor angiogenesis or metastasis, or it might be used for the dissolution of collagen IV-rich fibrotic growths, scars, or vasculature such as in treating varicose or spider veins. Promoting enzymatic 7S assembly may be useful for promoting vascularization during tissue regeneration.

### B. RECOMBINANT PRODUCTION

The Protomers may be produced by recombinant methods. Recombinant protein expression is commonly practiced for research and therapeutic purposes, and include the use of in vitro, bacterial, yeast, and mammalian culture expression systems. However, due to the complex protein folding that is required for the present invention to function properly, only certain mammalian expression systems are appropriate for the recombinant production of the invention. A description of such systems, as well as the general production methods, are presented below.

1. Mammalian Expression System

In additional to general protein expression mechanisms, the mammalian expression system much express specific chaperones and modifying enzymes in order to properly produce the invention. Specifically, the expression system should at minimum contain sufficient amounts of active prolyl-3-hydroxylase, prolyl-4-hydroxylase, lysyl hydroxylase, glycosylating enzymes, heat shock protein 47, protein secretion mechanisms, melanoma inhibitory activity member 3 (MIA3), and COPII.

In addition to the requirements detailed above, efficient production of the invention may occur under conditions that yield large amounts of recombinant product per unit of culture medium. Certain growth factors or molecules may be added to the culture conditions to enhance yield, such as TGFβ1, pyruvate, and glucose, depending on the expressing cell line. The invention is amenable to production in various systems, such as adherent or suspension cultures. Additionally, a variety of cell lines may be used to for expression including Chinese hamster ovary (CHO) cells, Cos7 cells, or other insect or mammalian cell lines. Optionally, to enhance yield or enzymatic modifications on the recombinant proteins, the expression system may be recombinantly engineered to co-express higher levels of one or more the required components listed above.

2. Purification and Manipulation of the Protomer

The protein may be expressed into the culture media and conjugated to commonly used purification tags, such as FLAG-tag or others. Importantly, when the recombinant proteins are expressed separately, purification of the individual proteins also occurs separately. Upon obtaining purified proteins, the are combined at the desired stoichiometry. For example, to assemble an α112 Protomer, twice as much α1 should be mixed with each proportion of α2; for assembling an α345, equal amounts of all proteins are combined. In order to control the assembly, all proteins should be combined in a low halide buffer, preferably 1 mM or lower. The protein purity and degree of assembly may be readily monitored via gel filtration chromatography or size exclusion chromatography. The inventors regularly use an S200 column (GE Healthcare) in 1× Tris-Buffered Saline when studying the Protomer.

The final conformation of the Protomer may be controlled, depending on the desired product. If isolated Protomers are desired, the material should be kept at room temperature or below, preferably 4° C., and the buffer system should be kept free of halogens or calcium.

If the desired product is a population of Protomers that are joined via sulfilmine bonds, then the sample should first be incubated for at least 24 hours in 100 mM or higher of a halide, preferably chloride. Subsequently, the protein should either be reacted with excess hypobromous acid or with a source of peroxidasin enzyme, Br⁻ ions, and oxidant source (such as H₂O₂). To purify the crosslinked product, the protein should be dialyzed into a halide-free buffer and the desired product purified by gel filtration chromatography or size exclusion chromatography.

### C. ANTIBODY PRODUCTION

1. General Methods

Antibodies to collagen IV may be produced by standard methods as are well known in the art (see, e.g, Antibodies: A Laboratory Manual, Cold Spring Harbor Laboratory, 1988; U.S. Pat. No. 4,196,265). The methods for generating monoclonal antibodies (MAbs) generally begin along the same lines as those for preparing polyclonal antibodies. The first step for both these methods is immunization of an appropriate host or identification of subjects who are immune due to prior natural infection. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions. One particular murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line. More recently, additional fusion partner lines for use with human B cells have been described, including KR12 (ATCC CRL-8658; K6H6/B5 (ATCC CRL-1823 SHM-D33 (ATCC CRL-1668) and HMMA2.5 (Posner et al., 1987). The antibodies in this invention were generated using the SP2/0/mIL-6 cell line, an IL-6 secreting derivative of the SP2/0 line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g, hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like.

The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g, a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the invention can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present invention include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

2. Antibodies

In on embodiment, the antibody is an Immunoglobulin G (IgG) antibody isotype. Representing approximately 75% of serum immunoglobulins in humans, IgG is the most abundant antibody isotype found in the circulation. IgG molecules are synthesized and secreted by plasma B cells. There are four IgG subclasses (IgG1, 2, 3, and 4) in humans, named in order of their abundance in serum (IgG1 being the most abundant). These range from having high to no affinity for the Fc receptor.

IgG is the main antibody isotype found in blood and extracellular fluid allowing it to control infection of body tissues. By binding many kinds of pathogens—representing viruses, bacteria, and fungi—IgG protects the body from infection. It does this via several immune mechanisms: IgG-mediated binding of pathogens causes their immobilization and binding together via agglutination; IgG coating of pathogen surfaces (known as opsonization) allows their recognition and ingestion by phagocytic immune cells; IgG activates the classical pathway of the complement system, a cascade of immune protein production that results in pathogen elimination; IgG also binds and neutralizes toxins. IgG also plays an important role in antibody-dependent cell-mediated cytotoxicity (ADCC) and intracellular antibody-mediated proteolysis, in which it binds to TRIM21 (the receptor with greatest affinity to IgG in humans) in order to direct marked virions to the proteasome in the cytosol. IgG is also associated with Type II and Type III Hypersensitivity. IgG antibodies are generated following class switching and maturation of the antibody response and thus participate predominantly in the secondary immune response. IgG is secreted as a monomer that is small in size allowing it to easily perfuse tissues. It is the only isotype that has receptors to facilitate passage through the human placenta. Along with IgA secreted in the breast milk, residual IgG absorbed through the placenta provides the neonate with humoral immunity before its own immune system develops. Colostrum contains a high percentage of IgG, especially bovine colostrum. In individuals with prior immunity to a pathogen, IgG appears about 24-48 hours after antigenic stimulation.

3. Engineering of Antibody Sequences

In various embodiments, one may choose to engineer sequences of the identified antibodies for a variety of reasons, such as improved expression, improved cross-reactivity, diminished off-target binding or abrogation of one or more natural effector functions, such as activation of complement or recruitment of immune cells (e.g, T cells). In particular, IgM antibodies may be converted to IgG antibodies. The following is a general discussion of relevant techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns. Recombinant full length IgG antibodies can be generated by subcloning heavy and light chain Fv DNAs from the cloning vector into a Lonza pConIgG1 or pConK2 plasmid vector, transfected into 293 Freestyle cells or Lonza CHO cells, and collected and purified from the CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

pCon Vectors™ are an easy way to re-express whole antibodies. The constant region vectors are a set of vectors offering a range of immunoglobulin constant region vectors cloned into the pEE vectors. These vectors offer easy construction of full length antibodies with human constant regions and the convenience of the GS System™.

Antibody molecules will comprise fragments (such as F(ab′), F(ab′)₂) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

It may be desirable to “humanize” antibodies produced in non-human hosts in order to attenuate any immune reaction when used in human therapy. Such humanized antibodies may be studied in an in vitro or an in vivo context. Humanized antibodies may be produced, for example by replacing an immunogenic portion of an antibody with a corresponding, but non-immunogenic portion (i.e., chimeric antibodies). PCT Application PCT/US86/02269; EP Application 184,187; EP Application 171,496; EP Application 173,494; PCT Application WO 86/01533; EP Application 125,023; Sun et al, 1987; Wood et al., 1985 and Shaw et al., 1988; all of which references are incorporated herein by reference. General reviews of “humanized” chimeric antibodies are provided by Morrison (1985); also incorporated herein by reference. “Humanized” antibodies can alternatively be produced by CDR or CEA substitution. Jones et al. (1986); Verhoeyen et al. (1988); Beidler et al. (1988); all of which are incorporated herein by reference.

Modified antibodies may be made by any technique known to those of skill in the art, including expression through standard molecular biological techniques, or the chemical synthesis of polypeptides. Methods for recombinant expression are addressed elsewhere in this document.

4. Expression

Nucleic acids according to the present disclosure will encode antibodies, optionally linked to other protein sequences. As used in this application, the term “a nucleic acid encoding a collagen IV antibody” refers to a nucleic acid molecule that has been isolated free of total cellular nucleic acid. Expression of antibodies can be effected in expression systems geared particularly toward recombinant production of antibodies, following the general methods of nucleic acid expression described elsewhere in this document.

### D. TISSUE DISEASE STATES AND DISORDERS

1. Collagen IV Diseases

An increasing number of human diseases are being associated with perturbation of collagen IV scaffolds. Genetic mutation of collagen IV can cause Alport's Syndrome, stroke, hearing loss, renal cysts, renal insufficiency, hematuria, retinal artery tortuosity or hemorrhage, anterior segment dysgenesis, congenital glaucoma optic nerve hyperplasia, cardia abnormalities including supraventricular arrhythmia, and structural defects in neural and vascular tissue (Kuo, Labelle-Dumais, and Gould, Hum. Mol. Genet., 2012). Genetic mutations found in proteins involved with the biosynthesis of collagen IV can cause osteogenesis imperfecta (type VIII, P3H1 mutations; type X, HSP47 mutations), myopia (P3H2 or PLOD3 mutation), cataracts (P3H2, PXDN, or PLOD3 mutations), retinal degeneration or detachment (P3H2 mutation), type VI Ehlers-Danlos syndrome (PLOD1 mutation), type 2 Bruck syndrome (PLOD2 mutation), deafness (PLOD3 mutation), flat facial profile (PLOD3 mutation), arterial rupture (PLOD3 mutation), osteopenia (PLOD3 mutation), joint contractures and fractures (PLOD3 mutations), skin blistering (PLOD3 mutations), and nail abnormalities (PLOD3 mutations) (Kuo, Labelle-Dumais, and Gould, Hum. Mol. Genet., 2012).

The present invention allows production of a recombinant therapeutic that, in one embodiment, can functionally replace missing collagen IV when administered to patients possessing one or two mutated collagen IV genes, such as Alport's patients. This same embodiment may also find utility in treating patients with genetic disease caused by mutated version of any enzyme that assists in the biosynthesis and/or assembly of collagen IV scaffolds.

In another embodiment, the invention may be used to treat patients whose basement membranes have been damaged through natural aging, oxidative stress, chemotherapy, radiation, or inflammation. These processes all hold potential of chemically modifying basement membranes, as well as collagen IV, such that the matrices and scaffolds are functionally compromised and provide a basis for disease. As such, the invention may effectively provide therapeutic replacement of endogenous collagen IV in such patients.

2. Diseases Impacting Collagen IV Binding Partners

Considering that collagen IV binds a diverse and numerous listing of proteins and glycoproteins, it therefore follows that collagen IV or similar molecules may be able to modulate the activity of said binding partners in vivo. Accordingly, particular embodiments of the invention may contain one or more binding sites for nidogen, usherin, fibronectin, laminin, chondroitin sulfate proteoglycan, heparin sulfate proteoglycan, factor IX, glycoprotein VI, heparin, heat shock protein 47, prolyl 3-hydroxylase, prolyl 4-hydroxylase, glycosyltransferase, Goodpasture antigen binding protein, bone morphogenic protein 4, transforming growth factor β type 1, osteonectin, collagen VII, decorin, integrin α111, integrin α2β1, integrin α3β1, integrin αVβ3, integrin αVβ5, discoidin domain receptor 1, discoidin domain receptor 2, or cluster of differentiation 47 (CD47). Additional embodiments may contain binding sites for two or more different binding partners of collagen IV, allowing the activity of multiple distinct binding partners to be simultaneously modulated.

Genetic insults to certain of these binding partners is reported as the basis for some rare diseases, as is the case then mutation in the gene for usherin (USH2A) cause Type II Usher Syndrome in humans. In healthy individuals, ushering is important for tissue development of the retina and inner ear. In one embodiment, the invention may be used for binding recombinant usherin protein and selectively delivering it to the specific tissue locations where it is needed most. Considering that the disclosed composition is capable of integrating with endogenous basement membranes, the delivery of usherin protein via the Protomer, as described above, may allow the therapeutic protein to be retained at the desired site and thereby potentially increase treatment efficacy.

As non-genetic example, expression of the integrin α1β1 has been suggested to be important for Kras-induced lung cancer (Macias-Perez et al., Cancer Res., 2008). Notably, the inventors have demonstrated the ability of this disclosed invention to selectively bind integrin receptors. As one potential application, this invention may be used as a medical treatment for Kras(+) cancers, for either systemic or localized administration. Here, the composition would contain an integrin α1β1 binding site, providing a preferred binding target for tumor cells. Some patients may benefit by simply interfering with normal integrin α1β1 binding, whereby the composition acts as a decoy receptor to interrupt the signaling activity of the tumor cell.

Alternatively, the invention may be used to deliver one or more desired binding partners to a target tissue. A particular advantage of this embodiment is found within the non-covalent nature of the binding interaction between the invention and the binding partner(s). This allows the binding partner(s) to be slowly released within the target tissue, with the rate of release being determined by the kinetics of the respective binding interaction. This may be accomplished by combining in solution the binding partner(s) with the invention, possessing one or more binding sites for the desired binding partner(s), then administering the combined solution to a patient. Optionally, a purification step may be added in between the mixing and administration steps.

In another preferred embodiment, the invention may be used to concentrate a desired binding partner within a particular tissue or site. This may be accomplished by administering the invention, possessing a binding site for the desired partner, to a patient such that the invention becomes bound within the basement membrane of the target tissue. Said invention should subsequently and selectively immobilize nearby endogenous or therapeutic molecules of the desired binding partner, effectively concentrating the binding partner near the target tissue.

Optionally, if deemed medically desirable, the invention may be conjugated to binding partner prior to administration to patients using standard methods of conjugating proteins and molecules. In this form, the invention may be used to deliver the desired binding partner to a target tissue in a manner that prevents said partner from diffusing away from the target tissue.

3. Cancer

The extracellular environment heavily influences the development and progression of cancerous cells. Often referred to generically as the influence of “extracellular matrix” (ECM), basement membranes and collagen IV scaffolds can strongly contribute to the development and spread of cancer cells. For many cancers, key developmental stages include but are not limited to maintenance of the cancer stem cell niche, the epithelial-mesenchymal transition, the invasiveness and subsequent circulation of cancer cells, and the development of metastatic secondary tumors. Basement membranes influence each of these stages, and in many cases, provide conditions that permit or even promote the progression of cancer cells through these stages (Borovski et al., Cancer Res., 2011). Such environmental influence occurs in the presence of any genetic mutations within the cancer cells.

Notably, there is even evidence that some cancer cells never progress into malignancies, allowing the host individual to live in a seemingly healthy state. Regarding these benign cancers, some prominent researchers hypothesized that conditions of the local ECM serve as a molecular restraint to prevent progression of the cancer (Bissell and Hines, Nat. Med., 2011).

Collagen IV has been shown to be a critical component in the development of some metastatic liver tumors in patients with colon cancer (Burnier et al., Oncogene, 2011). Intriguingly, at least one report has indicated that some colon cancer patients may also exhibit lowered blood concentrations of Br⁻ relative to healthy individuals (Shenberg et al., J. Trace Elements Med. Biol., 1995).

Basement membranes use a combination of mechanical properties and protein composition to exert their influence over cancer cells. Both factors have been shown to govern various aspects of cancer development including epithelial-mesenchymal transition and invasiveness. Importantly, collagen IV scaffolds are key to the mechanics as well as the composition of basement membranes, further reinforcing their role in cancer development.

a. Therapeutically Disrupting Basement Membranes to Treat Cancer

Considering that collagen IV scaffolds are central efforts of basement membrane stiffness, the present invention may be used to perturb the stability or assembly of basement membranes as a strategy for treating or preventing cancer. This may provide an efficient means for disrupting the stem cell nice of solid or hematologic tumors, hindering epithelial-mesenchymal transition, or preventing or delaying the development of metastatic or secondary tumors.

In one preferred embodiment, the invention may comprise an antibody that targets internal features of collagen IV NC1 trimers. In a preferred form of this embodiment, binding of the antibody to the NC1 trimers would prevent assembly of NC1 hexamers, thus impairing basement membrane assembly and leading to the destruction of the overlying tumorous tissue.

In another embodiment, the invention comprises a heterotrimeric recombinant protein that binds NC1 trimers within tumor basement membranes. In a preferred form of this embodiment, the composition lacks 7S domains and thus unable to form crosslinked 7S structures with nearby collagen IV protomers, resulting in instability within the basement membrane and destruction of the overlying tumorous tissue.

In yet another embodiment, the invention (1) binds NC1 trimers within tumor basement membranes and (2) is bound to a chemotherapeutic protein or molecule. In this case, the invention acts as a drug delivery device that selectively accumulates around the tumor.

In all cases, the term “tumor basement membrane” and “overlying tumorous tissue” may refer specifically to cancerous cells as well as, more generally, to non-cancerous cells that surround the tumor. For example, the invention may comprise an anti-angiogenesis treatment used to inhibit basement membrane assembly of the tumor vasculature. Alternatively, the invention may be used to modify an epithelial basement membrane in a region tissue deemed to be at risk of or suspected of harboring cancer stem cells or of undergoing an epithelial-mesenchymal transition, invasion, or other cancerous event.

b. Selectively Binding Cancer Cells

The invention may be used to reduce the number of circulating cancer cells. One readily apparent application of this would be to prevent metastasis by removing circulating metastatic cells in at-risk patients. In this case, the invention could administered into the patient's bloodstream where the invention would bind the cells and target them for destruction via immune, chemical, radiation, or other treatment. A preferred embodiment for this application would comprise one or more integrin binding domains within the recombinant hetero-triple helical protein.

Alternatively, the invention may be used in an extracorporeal manner by being covalently bound within a medical tube or filtering column. Upon passing the patient's blood through the tube or column, the target cells would be selectively removed via binding to the invention and the remaining purified blood returned to the patient. A preferred embodiment for this application would comprise one or more integrin binding domains within the recombinant hetero-triple helical protein.

4. Angiogenesis & Vascular Stability

Angiogenesis is the development of new vasculature, or blood vessels, within an organ or tissue. It is a requirement for tissue development, including tissue regeneration. However, it is also involved with various undesirable and pathologic conditions including tumor development and macular degeneration.

Angiogenesis is required for tissue development and as such, it is a key step during wound healing and tissue regeneration. Collagen IV scaffolds are critical to the stability of blood vessels, where destruction of the scaffold can result in deterioration of the overall vessel. Certain patient populations may benefit from collagen IV-based treatments that promote angiogenesis, such as individuals with chronic ischemic wounds or those in need of tissue regeneration. Excessive angiogenesis may be seen in cancer, age-related macular degeneration (the “wet” form), and possibly varicose veins.

a. Vascular Instability During Hemorrhagic Stroke and Aortic Aneurisms

Mutations in collagen IV have been shown to be the cause of some cases of stroke, particularly hemorrhagic stroke. In this case, damage to the α112 collagen IV network created instability within the vasculature which render the patient vulnerable to aneurisms. Notably, enzymatic degradation of collagen IV networks in the aorta, using the enzyme collagenase, is a means of inducing experimental aortic aneurisms. Together, this highlights the key role of collagen IV scaffolds in vascular physiology.

The disclosed invention may find utility as a therapeutic bioscaffold to treat individuals at risk of stroke or aneurism due to missing, damaged, or deteriorating collagen IV networks. Here, the invention could be manufactured as Protomers that activate upon entering the patient's bloodstream, binding at the site of injury or damage and effectively assembling into a synthetic replacement network that mimics certain features of collagen IV.

b. Goodpasture's Disease

Collagen IV sulfilimine bonds are implicated in the etiology of Goodpasture's Disease, an autoimmune condition characterized by autoantibodies that target collagen IV NC1 domains. Laboratory studies indicate that important autoepitopes on collagen IV are unreactive with autoantibodies when sulfilimine crosslinks are intact, likely due to conformational constraints imposed on collagen IV by the crosslink. Animal studies have shown that mice, which naturally possess abundant amounts of sulfilimine crosslinks, are largely immune to experimental Goodpasture's Disease.

A key etiologic event in clinical Goodpasture's Disease is believed to be perturbation of sulfilimine crosslinks, either via inhibiting their formation or disrupting existing bonds. In the absence of sulflimine crosslinks, the NC1 domain adopts a pathogenic conformation that is recognized by the disease auto-antibodies.

Consequently, the present innovation may be useful in treating Goodpasture's Disease. The goal of current treatments is to reduce the titer of circulating auto-antibodies that recognize collagen IV, yet typical treatment regimens deplete the patient of all circulating antibodies. Clearly, while this is effective, it may unnecessarily remove beneficial antibodies that protect the patient from infection. The composition described herein may be used as a medical device to selectively remove pathogenic auto-antibodies from circulation via extra-corporeal therapy. In this application, the invention may be immobilized within an absorber device. During treatment, patient's blood will be routed outside the body through a tube into the absorber, allowing pathogenic autoantibodies to bind the Protomer composition and thus be selectively removed before the bloodstream is routed back into the patient. Similar treatment strategies are employed for Pemphigus vulgaris and dilative cardiomyopathy. Thus, this particular embodiment may enable the standard techniques of absorber therapy to be applied in the context of treating Goodpasture's Disease.

### E. TREATMENT, PHARMACEUTICAL FORMULATIONS AND ROUTES OF ADMINISTRATION

The collagen IV agents of the present disclosure may be administered by a variety of methods, e.g, orally or by injection (e.g subcutaneous, intravenous, intraperitoneal, etc.).

Depending on the route of administration, the active compounds may be coated in a material to protect the compound from the action of acids and other natural conditions which may inactivate the compound. They may also be administered by continuous perfusion/infusion of a disease or wound site.

To administer the agents by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984).

The agents may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The agents can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

Active compounds are administered at a therapeutically effective dosage sufficient to treat a condition associated with a condition in a patient. A “therapeutically effective amount” preferably reduces the amount of symptoms of the condition in the infected patient by at least about 20%, more preferably by at least about 40%, even more preferably by at least about 60%, and still more preferably by at least about 80% relative to untreated subjects. For example, the efficacy of a compound can be evaluated in an animal model system that may be predictive of efficacy in treating the disease in humans, such as the model systems shown in the examples and drawings.

The actual dosage amount of an agent of the present disclosure or composition comprising an inhibitor of the present disclosure administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In certain embodiments, a pharmaceutical composition of the present disclosure may comprise, for example, at least about 0.1% of a compound of the present disclosure. In other embodiments, the compound of the present disclosure may comprise between about 2% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc.

1. Devices for Delivery of Therapeutic Compounds

The present invention involves, in some aspects, the provision of devices for delivery of collagen IV surrogates to wounds. In general, it is contemplated that any device or material that is brought into contact with a wound is a suitable vehicle for delivering collagen IV surrogates. The following devices/materials are exemplary in nature and are not meant to be limiting.

a. Wound Dressings

The present invention in one aspect, provides for various wound dressings that incorporate or have applied thereto the agents of the present disclosure. Dressings have a number of purposes, depending on the type, severity and position of the wound, although all purposes are focused towards promoting recovery and preventing further harm from the wound. Key purposes of are dressing are to seal the wound and expedite the clotting process, to soak up blood, plasma and other fluids exuded from the wound, to provide pain relieving effect (including a placebo effect), to debride the wound, to protect the wound from infection and mechanical damage, and to promote healing through granulation and epithelialization.

The following list of commercial dressings includes those that may be employed in accordance with the present invention: Acticoat, Acticoat 7, Actisorb Silver 220, Algisite M, Allevyn, Allevyn Adhesive, Allevyn Cavity, Allevyn Compression, Allevyn Heel, Allevyn Sacrum, Allevyn cavity wound dressing, Aquacel, Aquacel AG, Aquacel ribbon, Bactigras, Biatain Adhesive, Bioclusive, Biofilm, Blenderm, Blue line webbing, Bordered Granuflex, Calaband, Carbonet, Cavi-care, Cellacast Xtra, Cellamin, Cellona Xtra, Cellona elastic, Chlorhexitulle, Cica-Care, Cliniflex odour control dressing, Clinisorb odour control dressing, Coban, Coltapaste, Comfeel Plus, Comfeel Plus pressure relieving dressing, Comfeel Plus transparent dressing, Comfeel Plus ulcer dressing, Comfeel seasorb dressing, Comfeel ulcer dressing, Contreet Non-Adhesive, Crevic, Cutinova Hydro, Cutinova Hydro Border, Debrisan absorbent pad, Debrisan beads, Debrisan paste, Delta-Cast Black Label, Delta-Cast conformable, Delta-Lite S, Duoderm extra thin, Durapore, Elastocrepe, Elset/Elset ‘S’, Flamazine, Fucidin Intertulle, Geliperm granulated gel, Geliperm sheet, Granuflex (Improved formulation), Granuflex extra thin, Granugel, Gypsona, Gypsona S, Hypafix, Icthaband, Icthopaste, Inadine, Intrasite Gel, lodoflex, Iodosorb, Iodosorb ointment, Jelonet, K-Band, K-Lite, K-PLUS, Kaltocarb, Kaltostat, Kaltostat Fortex, Kaltostat cavity dressing, LarvE (sterile maggots), Lestreflex, Lyofoam, Lyofoam ‘A’, Lyofoam C, Mefix, Melolin, Mepiform, Mepilex, Mepilex AG, Mepilex Border, Mepilex Border Lite, Mepilex Border Sacrum, Mepilex Heel, Mepilex Lite, Mepilex Transfer, Mepitac, Mepitel, Mepore, Mepore Pro, Mesitran, Mesorb, Metrotop, Microfoam, Micropore, Opsite Flexigrid, Opsite IV 3000, Orthoflex, Oxyzyme, Paratulle, Polymem, Polymem Island & Shapes, Polymem Max, Polymem Silver, ProGuide, Profore, Promogran, Quinaband, Release, Scotchcast Plus, Scotchcast Softcast, Serotulle, Setopress, Silastic foam, Silicone N-A, Sofra-Tulle, Sorbsan, Sorbsan Plus, Sorbsan SA, Sorbsan Silver, Sorbsan Silver Plus Self Adhesive, Spenco 2nd Skin, Spyroflex, Spyrosorb, Tarband, Tegaderm, Tegaderm Plus, Tegagel, Tegapore, Tegasorb, Telfa, Tensopress, Tielle, Tielle Lite, Tielle Plus, Tielle Plus Borderless, Transpore, Unitulle, Veinoplast, Veinopress, Versiva, Vigilon, Viscopaste PB7, Xelma, and Zincaband.

A typical (sterile) dressing is one made of a film, foam, semi-solid gel, pad, gauze, or fabric. More particularly, sterile dressings are made of silicone, a fibrin/fibrinogen matrix, polyacrylamide, PTFE, PGA, PLA, PLGA, a polycaprolactone or a hyaluronic acid, although the number and type of materials useful in making dressings is quite large. Dressing may further be described as compression dressings, adherent dressing and non-adherent dressings.

Dressings may advantageously include other materials—active or inert. Such materials include gelatin, silver, cellulose, an alginate, collagen, a hydrocolloid, a hydrogel, a skin substitute, a wound filler, a growth factor, an antibody, a protease, a protease inhibitor, an antibacterial peptide, an adhesive peptide, a hemostatic agent, living cells, honey, nitric oxide, a corticosteroid, a cytotoxic drug, an antibiotic, an antimicrobial, an antifungal, an antiseptic, nicotine, an anti-platelet drug, an NSAID, colchicine, an anti-coagulant, a vasoconstricting drug or an immunosuppressive.

Wound dressings may also be part of a larger device, such as one that permits fixation of the dressing to a wound, such as an adhesive or a bandage. Dressings/devices may also include other features such as a lubricant, to avoid adhesion of the dressing to the wound, an absorber to remove seepage from the wound, padding to protect the wound, a sponge for absorbance or protection, a wound veil, an odor control agent, and/or a cover.

The collagen IV agent, or any other agent, may be applied to a dressing, or disposed in a dressing, by virtue of its introduction into or onto the dressing in a liquid, a salve, an ointment, a gel or a powder. Alternatively, the collagen IV agent or other agent may be added to a discrete element of a dressing (a sheet or film) that is included in the dressing during its manufacture.

Devices may also include a port, such as one providing operable connection between said sterile dressing and a tube, as well as a cover providing an airtight seal to or around a wound surface. Such embodiments are particularly useful in negative pressure wound therapy methods and devices.

b. Sutures

A surgical suture is a medical device used to hold body tissues together after an injury or surgery. It generally a length of thread, and it attached to a needle. A number of different shapes, sizes, and thread materials have been developed over time. The present invention envisions the coating or impregnating of sutures with agents of the present disclosure.

The first synthetic absorbable was based on polyvinyl alcohol in 1931. Polyesters were developed in the 1950s, and later the process of radiation sterilization was established for catgut and polyester. Polyglycolic acid was discovered in the 1960s and implemented in the 1970s. Today, most sutures are made of synthetic polymer fibers, including the absorbables polyglycolic acid, polylactic acid, and polydioxanone as well as the non-absorbables nylon and polypropylene. More recently, coated sutures with antimicrobial substances to reduce the chances of wound infection have been developed. Sutures come in very specific sizes and may be either absorbable (naturally biodegradable in the body) or non-absorbable. Sutures must be strong enough to hold tissue securely but flexible enough to be knotted. They must be hypoallergenic and avoid the “wick effect” that would allow fluids and thus infection to penetrate the body along the suture tract.

All sutures are classified as either absorbable or non-absorbable depending on whether the body will naturally degrade and absorb the suture material over time. Absorbable suture materials include the original catgut as well as the newer synthetics polyglycolic acid (Biovek), polylactic acid, polydioxanone, and caprolactone. They are broken down by various processes including hydrolysis (polyglycolic acid) and proteolytic enzymatic degradation. Depending on the material, the process can be from ten days to eight weeks. They are used in patients who cannot return for suture removal, or in internal body tissues. In both cases, they will hold the body tissues together long enough to allow healing, but will disintegrate so that they do not leave foreign material or require further procedures. Occasionally, absorbable sutures can cause inflammation and be rejected by the body rather than absorbed.

Non-absorbable sutures are made of special silk or the synthetics polypropylene, polyester or nylon. Stainless steel wires are commonly used in orthopedic surgery and for sternal closure in cardiac surgery. These may or may not have coatings to enhance their performance characteristics. Non-absorbable sutures are used either on skin wound closure, where the sutures can be removed after a few weeks, or in stressful internal environments where absorbable sutures will not suffice. Examples include the heart (with its constant pressure and movement) or the bladder (with adverse chemical conditions). Non-absorbable sutures often cause less scarring because they provoke less immune response, and thus are used where cosmetic outcome is important. They must be removed after a certain time, or left permanently.

In recent years, topical cyanoacrylate adhesives (“liquid stitches”) have been used in combination with, or as an alternative to, sutures in wound closure. The adhesive remains liquid until exposed to water or water-containing substances/tissue, after which it cures (polymerizes) and forms a flexible film that bonds to the underlying surface. The tissue adhesive has been shown to act as a barrier to microbial penetration as long as the adhesive film remains intact. Limitations of tissue adhesives include contraindications to use near the eyes and a mild learning curve on correct usage.

Cyanoacrylate is the generic name for cyanoacrylate based fast-acting glues such as methyl-2-cyanoacrylate, ethyl-2-cyanoacrylate (commonly sold under trade names like Superglue™ and Krazy Glue™) and n-butyl-cyanoacrylate. Skin glues like Indermil® and Histoacryl® were the first medical grade tissue adhesives to be used, and these are composed of n-butyl cyanoacrylate. These worked well but had the disadvantage of having to be stored in the refrigerator, were exothermic so they stung the patient, and the bond was brittle. Nowadays, the longer chain polymer, 2-octyl cyanoacrylate, is the preferred medical grade glue. It is available under various trade names, such as LiquiBand®, SurgiSeal®, FloraSeal®, and Dermabond®. These have the advantages of being more flexible, making a stronger bond, and being easier to use. The longer side chain types, for example octyl and butyl forms, also reduce tissue reaction.

c. Negative Pressure Wound Therapy

Negative pressure wound therapy (NPWT), also known as topical negative pressure, sub-atmospheric pressure dressings or vacuum sealing technique, is a therapeutic technique used to promote healing in acute or chronic wounds, fight infection and enhance healing of burns. A vacuum source is used to create sub-atmospheric pressure in the local wound environment. The wound is sealed to prevent dehiscence with a gauze or foam filler dressing, and a drape and a vacuum source applies negative pressure to the wound bed with a tube threaded through the dressing. The vacuum may be applied continuously or intermittently, depending on the type of wound being treated and the clinical objectives. Intermittent removal of used instillation fluid supports the cleaning and drainage of the wound bed and the removal of infectious material.

NPWT has multiple forms which mainly differ in the type of dressing used to transfer NPWT to the wound surface, and include both gauze and foam. Gauze has been found to effect less tissue ingrowth than foam. The dressing type depends on the type of wound, clinical objectives and patient. For pain sensitive patients with shallow or irregular wounds, wounds with undermining or explored tracts or tunnels, and for facilitating wound healing, gauze may be a better choice for the wound bed, while foam may be cut easily to fit a patient's wound that has a regular contour and perform better when aggressive granulation formation and wound contraction is the desired goal. The technique is often used with chronic wounds or wounds that are expected to present difficulties while healing (such as those associated with diabetes or when the veins and arteries are unable to provide or remove blood adequately).

d. Transdermal Delivery

Certain embodiments of the present invention pertain to transdermal or transcutaneous delivery devices for delivery of agents of the present disclosure. The therapeutic agent is embedded in or in contact with a surface of the patch. The patch can be composed of any material known to those of ordinary skill in the art. Further, the patch can be designed for delivery of the therapeutic agent by application of the patch to a body surface of a subject, such as a skin surface, the surface of the oral mucosa, a wound surface, or the surface of a tumor bed. The patch can be designed to be of any shape or configuration, and can include, for example, a strip, a bandage, a tape, a dressing (such as a wound dressing), or a synthetic skin. Formulations pertaining to transdermal or transcutaneous patches are discussed in detail, for example, in U.S. Pat. Nos. 5,770,219, 6,348,450, 5,783,208, 6,280,766 and 6,555,131, each of which is herein specifically incorporated by reference into this section and all other sections of the specification.

In some embodiments, the device may be designed with a membrane to control the rate at which a liquid or semi-solid formulation of the therapeutic agent can pass through the skin and into the bloodstream. Components of the device may include, for example, the therapeutic agent dissolved or dispersed in a reservoir or inert polymer matrix; an outer backing film of paper, plastic, or foil; and a pressure-sensitive adhesive that anchors the patch to the skin. The adhesive may or may not be covered by a release liner, which needs to be peeled off before applying the patch to the skin. In some embodiments, the therapeutic agent is contained in a hydrogel matrix.

Topical patch formulations may include a skin permeability mechanism such as: a hydroxide-releasing agent and a lipophilic co-enhancer; a percutaneous sorbefacient for electroporation; a penetration enhancer and aqueous adjuvant; a skin permeation enhancer comprising monoglyceride and ethyl palmitate; stinging cells from cnidaria, dinoflagellata and myxozoa; and/or the like. Formulations pertaining to skin permeability mechanisms are discussed in detail, for example, in U.S. Pat. Nos. 6,835,392, 6,721,595, 6,946,144, 6,267,984 and 6,923,976, each of which is specifically incorporated by reference into this section of the specification and all other sections of the specification. Also contemplated is microporation of skin through the use of tiny resistive elements to the skin followed by applying a patch containing adenoviral vectors as referenced by Bramson et al. (2003), and a method of increasing permeability of skin through cryogen spray cooling as referenced by Tuqan et al. (2005), and jet-induced skin puncture as referenced by Baxter et al. (2005), and heat treatment of the skin as referenced by Akomeah et al. (2004), and scraping of the skin to increase permeability.

In other embodiments, the patch is designed to use a low power electric current to transport the therapeutic agent through the skin. In other embodiments, the patch is designed for passive drug transport through the skin or mucosa. In other embodiments, the device is designed to utilize iontophoresis for delivery of the therapeutic agent.

The device may include a reservoir wherein the therapeutic agent is comprised in a solution or suspension between the backing layer and a membrane that controls the rate of delivery of the therapeutic agent. In other embodiments, the device includes a matrix comprising the therapeutic agent, wherein the therapeutic agent is in a solution or suspension dispersed within a collagen matrix, polymer, or cotton pad to allow for contact of the therapeutic agent with the skin. In some embodiments, an adhesive is applied to the outside edge of the delivery system to allow for adhesion to a surface of the subject.

In some embodiments, the device is composed of a substance that can dissolve on the surface of the subject following a period of time. For example, the device may be a file or skin that can be applied to the mucosal surface of the mouth, wherein the device dissolves in the mouth after a period of time. The therapeutic agent, in these embodiments, may be either applied to a single surface of the device (i.e., the surface in contact with the subject), or impregnated into the material that composes the device.

In some embodiments, the device is designed to incorporate more than one therapeutic agent. The device may comprise separate reservoirs for each therapeutic agent, or may contain multiple therapeutic agents in a single reservoir.

Further, the device may be designed to vary the rate of delivery of the therapeutic agent based on bodily changes in the subject, such as temperature or perspiration. For example, certain agents may be comprised in a membrane covering the therapeutic agent that respond to temperature changes and allow for varying levels of drug to pass through the membrane. In other embodiments, transdermal or transcutaneous delivery of the therapeutic agent can be varied by varying the temperature of the patch through incorporation of a temperature-control device into the device.

In preparing a transdermal patch according to the teachings of the specification and the knowledge of those skilled in the art, the collagen IV surrogate, an adhesive, and a permeation enhancer may be mixed together and dispensed onto a siliconized polyester release liner (Release Technologies, Inc., W. Chicago, Ill.). For example the transdermal patch formulation may consist of approximately 88% by composition of an acrylic copolymer adhesive, 2% of a nucleic acid expression construct, and 10% of a sorbitan monooleate permeation enhancer such as ARACEL 80® (ICI Americas, Wilmington, Del.). The mixture may then be dried and stored for treatment of a subject.

2. Combination Therapy

In addition to being used as a monotherapy, the compounds of the present disclosure may also find use in combination therapies. Effective combination therapy may be achieved with a single composition or pharmacological formulation that includes both agents, or with two distinct compositions or formulations, at the same time, wherein one composition includes a compound of this invention, and the other includes the second agent(s). Alternatively, the therapy may precede or follow the other agent treatment by intervals ranging from minutes to months.

Various combinations may be employed, such as where the collagen IV surrogate is “A” and “B” represents a secondary agent, non-limiting examples of which are described below:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the agents of the present disclosure to a patient will follow general protocols for the administration of pharmaceuticals, taking into account the toxicity, if any, of the drug. It is expected that the treatment cycles would be repeated as necessary.

Secondary agents include chloride, bromide, peroxide, molecular oxygen, electron-accepting compound such as flavin adenine dinucleotide (FAD), hypobromous acid, nicotinamide adenine dinucelotide (NAD & NADH), nicotinamide adenine dinucelotide phosphate (NADP & NADPH), inosine monophosphate (IMP), guanosine monophosphate (GMP) or a combination thereof.

### F. EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Materials & Methods

Materials.

Cell culture reagents were purchased from CellGro (Mediatech, Manassas, Va.), while all other chemicals and reagents were purchased from Sigma-Aldrich (Saint Louis, Mo.).

Methods. Preparation of Collagen IV NC1 and PXDN:

NC1 hexamers were isolated from tissues as described previously (Boutaud et al., 2000). Briefly, matrices were washed successively with buffered 1% sodium deoxycholate, then buffered 1 M NaCl, and finally low salt buffer prior to digestion with bacterial collagenase. Hexamers were purified from the digest using DE-52 cellulose and SEC chromatography (GE Life Sciences; Piscataway, N.J.). Alternatively, collagen IV was expressed in PFHR9 cell cultures in the presence of either 50 μM phloroglucinol or 1 mM KI to inhibit sulfilimine crosslink formation (Bhave et al., 2012), and hexamers isolated similarly to tissue-derived matrices. Recombinant PXDN was produced and purified as previously described (Bhave et al., 2012).

Dissociation and Assembly of NC1 Hexamers.

For dissociation, uncrosslinked hexamers were extensively dialyzed into various low-Cl buffer systems at 4° C., with the product being monitored using SEC. For hexamer assembly, dissociated NC1 domains in Tris-Ac were concentrated prior to the addition of NaCl and allowed to assemble at 37° C. for 24 hours. SEC was performed using an S200 sepharose column (GE Life Sciences; Piscataway, N.J.) on an AKTA FPLC with Unicorn software (GE Life Sciences; Piscataway, N.J.). Profiles were generated in SigmaPlot (Version 10).

Production of Mini-Protomers.

DNA constructs from the wild-type α1 and α2 sequences, encoding the NC1 domain and 84 GXY repeats. The CB3-derived α1β2 integrin binding site was incorporated via site mutagenesis. Recombinant constructs were expressed in HEK293 with G418 selection, and alternatively in SF9 cells. Protein products were purified via anti-FLAG affinity chromatography and SEC.

Solid-State I-Domain Binding Assay.

Mini-protomers and rat tail collagen I (BD Biosciences) were separately coated onto Nunc Maxisorp microtiter plates (Thermo Scientific), blocked with BSA, and probed with recombinant integrin alpha I-domains. The I-domains were detected with GST-conjugated primary antibodies and anti-GST-HRP secondary antibodies. Non-specific binding was measured in the presence of EDTA.

HT1080 Adhesion Assay.

Microtiter plates were coated as for solid-state binding assays prior to incubation with 1×10⁵ HT1080 cell/well for 1 hour with and without monoclonal antibodies against α1β2 integrin (MAB1998Z, Chemicon International). Unbound cells were washed out with 1×PBS while adherent cells fixed and stained with 0.1% crystal violet (Kueng, Silber, and Eppenberger, 1989)

In Vitro Crosslinking by PXDN or HOBr.

HOBr was synthesized by reacting sodium hypochlorite with excess Br− at high pH as described previously (McCall et al., 2014), then diluted into 10 mM phosphase buffer (pH 7.4) to create HOBr via protonation. Uncrosslinked hexamers were reacted with either HOBr or PXDN at 37° C. and the appropriate cofactors, and analyzed via 12% non-reducing SDS-PAGE gels and/or SEC.

Molecular Modeling and Molecular Dynamics (MD) Simulations.

Molecular models and MD simulations were based on the X-ray crystal structure of the bovine placenta collagen IV NC1 hexamer (1T61) (Vanacore et al., 2004). Molecular modeling was performed with PYMOL (Schrodinger, LLC). Protein binding surfaces were analyzed using LIGPLOT+ (Wallace et al., 1995) and the INTERSURF (Ray et al., 2005) algorithm of CHIMERA (Pettersen et al., 2004). AMBER 12 (Case et al., 2005) using ff99SB parameter sets (Cornell et al., 1996; Hornak et al., 2006) were used for MD simulations of NC1 hexamers, trimers, and monomers in 0 mM and 150 mM Cl− environments.

Multiple Sequence Alignment.

Sequences were obtained from Genbank and alignments were generated with GENEIOUS v.4.8.5 using the “blosum62” algorithm.

### Example 2—Results

Introduction.

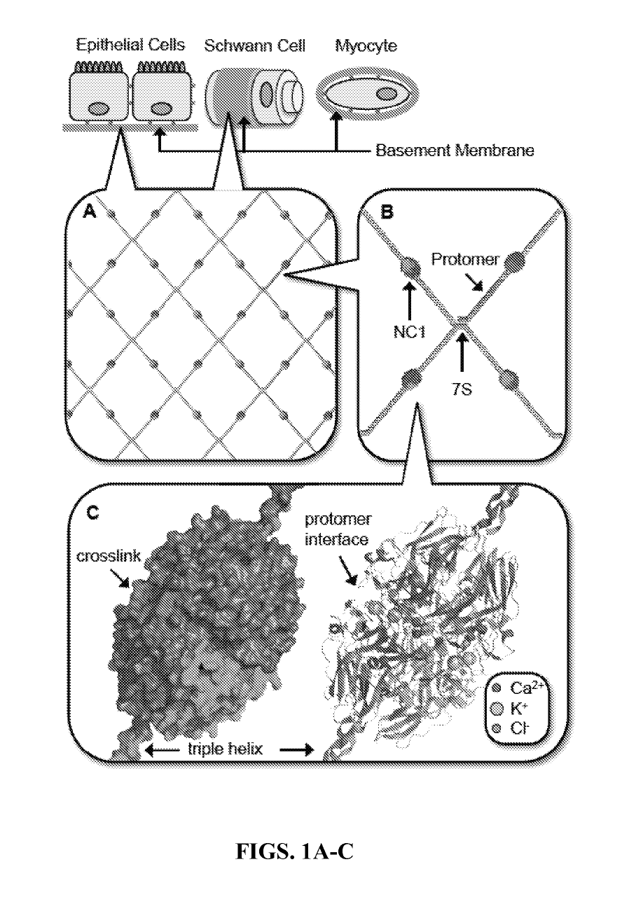

The extracellular microenvironment plays a pivotal role in tissue genesis, architecture and function. A core feature of these microenvironments is the basement membrane (BM), a specialized form of extracellular matrix that underlies epithelial (Daley and Yamada, 2013; Hagios et al., 1998; Hynes, 2009; Lu et al., 2012; Yurchenco, 2011) and endothelial cells (Rhodes and Simons, 2007), and ensheaths muscle (Campbell and Stull, 2003; Sanes, 2003), fat (Sillat et al., 2012), Schwann (Court et al., 2006) and decidua cells (Farrar and Carson, 1992; Wewer et al., 1985) (FIGS. 1A-C). BMs are fundamental components of the cellular toolkit that function as supramolecular scaffolds in sculpting diverse tissue architectures and functions. Known BM functions include compartmentalizing and providing structural integrity of tissues, guiding cell migration and adhesion delineating apical-basal polarity modulating cell differentiation during development, orchestrating cell behavior in tissue repair after injury, and guiding pluripotent cells to regenerate whole organs from de-cellularized BMs (Hynes, 2009, 2012; Yurchenco, 2011).

At the molecular level, BM scaffolds are comprised of collagen IV, laminin and proteoglycans that interlinked into a complex structure, collectively interacting with numerous other components. Collagen IV is a staple component of BMs, being observed as a supramolecular network in which collagen IV protomers, long triple-helical molecules, are connected end-to-end (FIG. 1B). Functionally, collagen IV networks provide a structural framework for the binding of integrins, for cell adhesion and signaling; binding BMPs (12-14), for signaling gradients during tissue development; and tethering a diverse assortment of extracellular molecules. Further, the collagen network provides tensile strength to BMs. Mutations in collagen IV cause BM destabilization and tissue dysfunction in humans, nematodes, flies, and mice. The clinical consequences of disrupting collagen IV networks include Alport syndrome, a genetic disorder resulting in renal failure, as well as various neurologic and vascular disorders (Gould et al., 2006) (Kuo, Labelle-Dumais, Gould, 2012). Collectively, without properly formed collagen IV networks, the BM scaffold is nonfunctional.

Collagen IV acts through the complex structural features encoded in its supramolecular network. For example, integrins bind within the triple helical motif of collagen IV protomers, contacting residues from two independent α-chains which requires proper chain register (Emsley et al., 2000; Kern et al., 1993). Within the network two protomers interact through their trimeric NC1 domains forming a NC1 hexamer at the interface (FIGS. 1A-C) and four protomers interact through their 7S domains forming 7S tetramers at the N-termini. The NC1 timer-trimer interface is reinforced by sulfilimine crosslinks formed by peroxidasin and bromide ions (Bhave et al., 2012; McCall et al., 2014). Perturbation of either peroxidasin or Br− limits the degree of crosslinking, disrupts tissue architecture, and causes early lethality in Drosophila. Indeed, its conservation from cnidarians to humans suggests the crosslink is a basic requirement for complex tissue development (Fidler et al., 2014), analogous to nutrient delivery, likely due to the unique structural reinforcement it provides the C-terminal NC1 hexamers.

Limited information is available regarding the mechanisms of collagen IV network assembly and the molecular pathogenesis triggered by genetic mutations. Assembly has been dogmatically understood to initiate with the intracellular formation of protomers, from individual collagen IV α chains, followed by extracellular assembly of protomers into networks. The NC1 domain has been long hypothesized to play a central role with chain selection and protomer nucleation, selecting from six α-chains for assembly into three distinct triple helical protomers (α121, α345, α565), presumably within with endoplasmic reticulum. After secretion into nascent BMs, the NC1 is thought to further guide the selective assembly of protomers into networks. Snapshots of network assembly have been seen via the crystal structure of the NC1 hexamer, the capacity of recombinant NC1 monomers to selectively assemble into hexamers, and the refolding of triple helix emanating from a NC1 hexamer. While this information is supportive, it remains circumstantial regarding the authentic role of NC1 domains in the assembly processes and provides little information about how collagen IV networks are assembled outside of the cell.

Studies using X-ray crystallography revealed that Cl⁻, K⁺, and Ca²⁺ ions are juxtaposed at the interface of two adjoining protomers (FIG. 1C). Herein, the inventors sought to test the hypothesis that these ions actively promote network assembly. Using recombinant technology to generate triple helical protomers, they demonstrate that NC1 domains direct protomer assembly as well as network assembly. Moreover, they discovered a Cl-mediated molecular switch within the NC1 domain that induces the extracellular formation of networks. Key residues are found in a broader range of organisms than sulfilimine crosslinks, implying that Cl⁻ is essential for BM formation. These discoveries provide fundamental insights into mechanisms of assembly of the collagen IV networks of BM scaffolds.

Cl− Induces Collagen IV NC1 Hexamer Assembly.

X-ray structures revealed specific ions along the protomer-protomer and the monomer-monomer interfaces of NC1 domains (FIG. 1C). The inventors hypothesized these ions may be mechanistically important for collagen IV network assembly. In the current study, the inventors used NC1 hexamers isolated from native basement membrane (bLBM) or extracellular matrix deposited by PFHR-9 cell line in culture as model systems to decipher the larger implications for network assembly. While the former system provides significant amount of authentic NC1 hexamer composed predominantly of monomers, the later system provides additional advantage of controlled perturbation of the NC1 domain crosslinking within hexamers using peroxidasin inhibitors as the inventors demonstrated previously (Bhave et al., 2012).

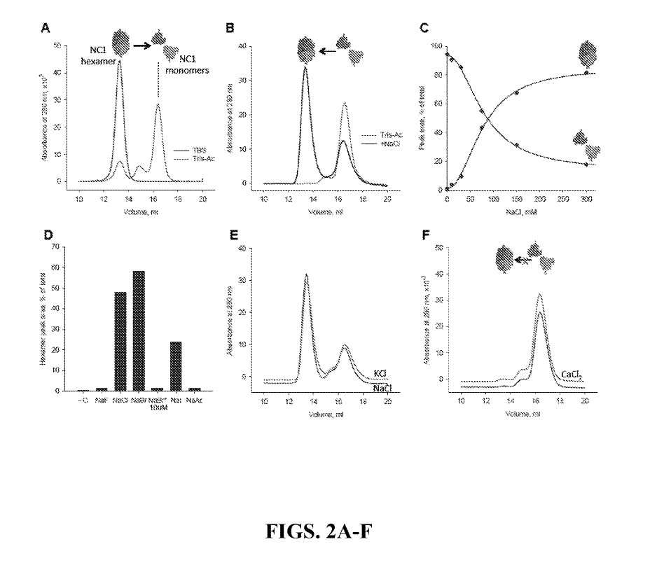

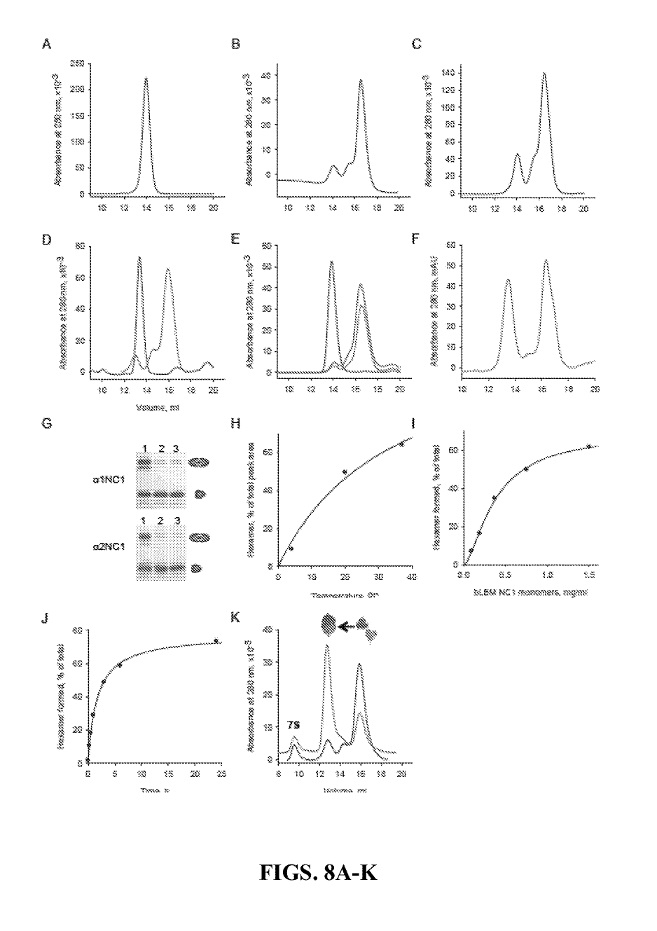

To explore the putative role of these ions, the inventors dialyzed NC1 hexamers from lens capsule basement membrane (LBM) from TBS into Tris-acetate buffer (TrisAc). This treatment caused the dissociation of hexamers into NC1 monomers as detected by the appearance of characteristic slower migrating peak by size-exclusion chromatography (SEC, FIG. 2A). Notably, similar dissociation could be achieved by treatment with strong protein denaturants including guanidine as well as urea (FIGS. 8A-C). Moreover, dialysis of uncrosslinked hexamer from PFHR9 cells into TrisAc also induced strong dissociation into monomers (FIG. 8D). This effect required the absence of NaCl while significant dissociation of LBM and PFHR9 hexamers was seen after dialysis into phosphate buffer (FIGS. 8E-F), further suggesting a stabilizing role for the ionic salt.

Next, the inventors asked whether specific ions could trigger the reverse process of collagen IV hexamer assembly. α1 and α2NC1 monomers were isolated from dissociated LBM hexamer, which had been prepared by dialysis into TrisAc and SEC fractionation (FIG. 2A). The monomers were concentrated, mixed at a 2:1 ratio of α1 and α2, and finally incubated with 100 mM NaCl at 37° C. This yielded an SEC peak that was indistinguishable from the authentic LBM hexamer and contained α1 as well as α2NC1 domains (FIG. 2B, FIG. 8G). The yield of reassembled hexamer was dependent on NaCl concentration, reaching saturation around 200 mM (FIG. 2C). In addition, incubation temperature and protein concentration both had a strong effect on hexamer assembly (FIGS. 8A-K. FIGS. 8H-I). The assembly reaction deplayed slow kinetics even under optimal in vitro conditions, reaching equilibrium in 24 hrs (FIG. 8J). NC1 domains isolated from PFHR9 cells similarly reassembled into hexamers in the presence of NaCl (FIG. 8K).

The inventors sought to determine which ion, Na⁺ or Cl⁻, was inducing the observed hexamer assembly. To this end, the inventors further explored reassembly of LBM hexamer in the presence of various monovalent anions. Among the halides only Cl⁻ and Br⁻ strongly induced hexamer formation, while I− was significantly less efficient, and F⁻ did not induce hexamer assembly at 100 mM (FIG. 2D). Noting that Br− triggered assembly at 100 mM, above the generally-recognized toxic level of ca. 12 mM (van Leeuwen and Sangster, 1987), the inventors tested the physiologically relevant concentration of 100 μM Br⁻, which was unable to induce hexamer assembly (FIG. 2D).

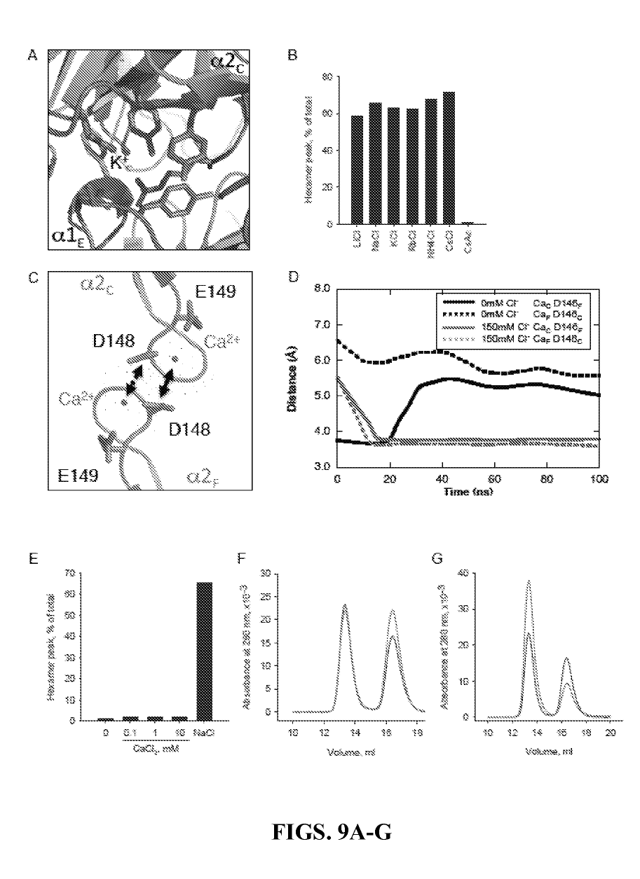

In contrast to anions, no specific cations was observed in assembly (FIGS. 9A-G). K⁺ acted similarly to Na⁺ when tested in chloride form (FIG. 2E), and the larger monovalent cations cesium and ammonium were also comparable (FIGS. 9A-G). Modeling studies of the cation binding site suggest that the plane of the aromatic side chains is orthogonal to the crystallographic location of the potassium cation (FIG. 9A). Intriguingly, four of the seven cation contact residues are located on the β-hairpin suggesting they may be involved with NC1:NC1 interactions, yet their role remains ambiguous.

Calcium is a well-known cation that binds and induces conformational changes in many proteins (Chou et al., 2001). The calcium binding site is located within the interior hexamer cavity where it coordinates residues D148 and E149 of the α2 monomers (FIG. 9C), potentially modifying hexamer assembly. However, a physiological concentration of Ca2+ alone did not induce assembly (FIG. 2F), and Cl-mediated assembly proceeded efficiently even in the presence of EDTA (FIGS. 9A-G). The inventors observed an apparent increase of hexamer yield when Ca²⁺ and Cl⁻ were provided together, indicating that Ca+ may potentiate the activity of Cl− (FIG. 9G). MD simulations predict that chloride in the bulk solvent enhances the inter-protomer association of Ca2+ and D148 (FIG. 9D).

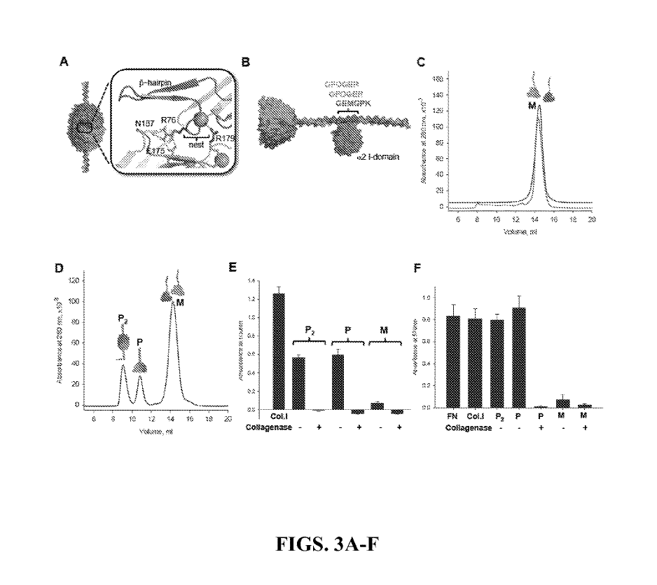

Taken together, the inventors concluded that Cl⁻ is the key anion required for hexamer assembly. They noticed that Cl⁻ binds within the crystal structure near specific salt bridges that span the trimer:trimer interface (FIG. 3A). Hypothesizing that Cl⁻ provides a molecular signal which triggers hexamer assembly, the inventors sought to develop suitable reagents that would enable us to elucidate the underpinning mechanism of the observed Cl− activity.

Production and Characterization of Recombinant Protomers.

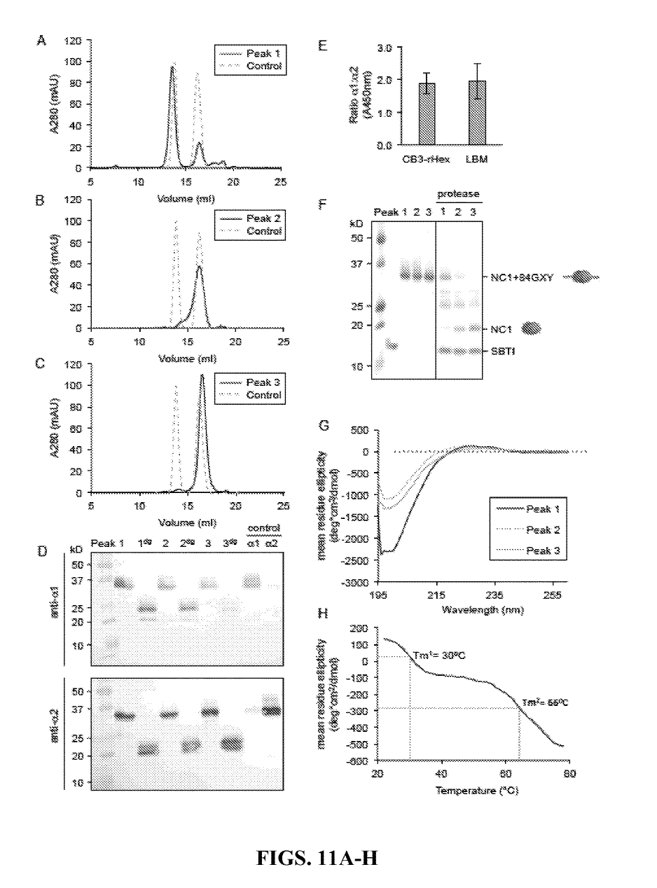

In order to rigorously examine the NC1 assembly mechanisms within collagen IV scaffolds and the pivotal role of Cl⁻, the inventors recognized the need for a new strategy of obtaining collagen IV protomers. To this end, they utilized novel truncated α112 protomers (r-Prot) which they designed and recombinantly produced (FIGS. 3A-F). The inventors designed each construct to contain an NC1 domain that was adjacent to a collagenous domain encoding 28 GXY-repeats, corresponding to the C-terminal region of native α112 collagen IV protomers. Individual α1 and α2 constructs were expressed in HEK 293 cells and purified in monomeric form by SEC (FIG. 3C). After incubation of the concentrated monomers at a 2:1 ratio (α1:α2) in the presence of Cl−, two distinct lower mobility peaks were formed and resolved by SEC. The first peak eluted at 9 ml (FIG. 3D). Following bacterial collagenase digestion, it produced a peak identical to the native LBM hexamer (FIG. 11A) and was thus identified as protomer dimer (P2). Moreover, it contained α1 and α2 NC1 domains at a 2:1 ratio as quantified by ELISA, which is identical to the stoichiometry of native LBM hexamers (FIG. 11E). The second peak eluted at 11 ml (FIG. 3D), was converted to NC1 monomers by collagenase digestion (FIG. 11B). The inventors concluded that this comprised r-Prot (P). As this was the first evidence of an isolated NC1 trimer, it suggests that lateral association between NC1 domains per se are too weak to produce a stabile trimer, underscoring the requirement for the triple-helical domain to stabilize the protomer. A population of monomeric chains were still present following incubation (FIG. 3D), which was converted to NC1 domains by collagenase (FIG. 11C).

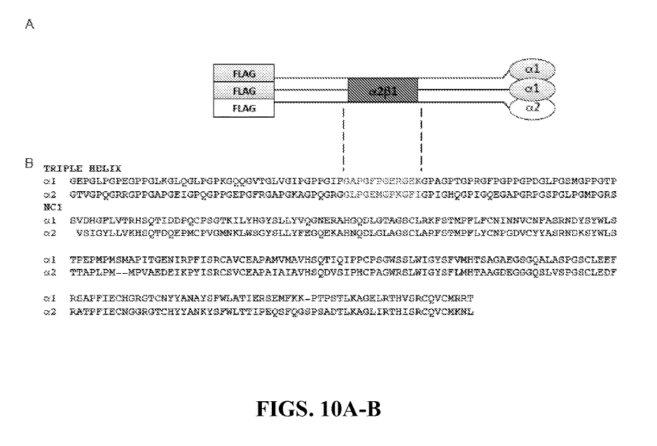

To access the structural competence of the triple-helical domain, the inventors incorporated an α2β1 integrin binding site derived from CB3 region of native collagen IV in the middle part of collagenous domain (FIG. 3A, FIGS. 10A-B). Formation of the triple helical collagenous domain in P and P2 was confirmed by the resistance of both forms to limited proteolysis (FIG. 11D), as well as circular dichroism spectrometry (FIGS. 11G-H) which yielded a characteristic positive peak at 220 nm and melting temperature (Tm) of 30° C. Both r-Prot and r-Prot dimers, isolated by SEC, bound the α2 integrin I-domain in Mg²⁺-dependent manner while monomers were inactive (FIG. 3E). Collagenase treatment completely eliminated activity, confirming that binding occurred only at the triple helix (FIG. 3E). The inventors further tested binding activity in cell adhesion assays with HT1080 cells (Eble, Kuhn). Cells adhered to both r-Prot and r-Prot dimers, but not monomers, while collagenase pretreatment prevented binding (FIG. 3F). Further inhibition of HT-1080 cell adhesion was neutralized with function-blocking monoclonal antibodies against α2β1 integrin (FIGS. 11A-H).

In sum, the truncated protomers faithfully reproduced the key elements of native collagen IV protomers as designed, including a properly folded NC1 trimer capable of forming hexamers as well as a functional triple helix with correct folding, registration, and stoichiometry. With these reagents in hand, the inventors the inventors sought to interrogate the Cl-triggered mechanism of collagen IV scaffold assembly.

Protomers self-assemble while network self-assembly requires Cl⁻.

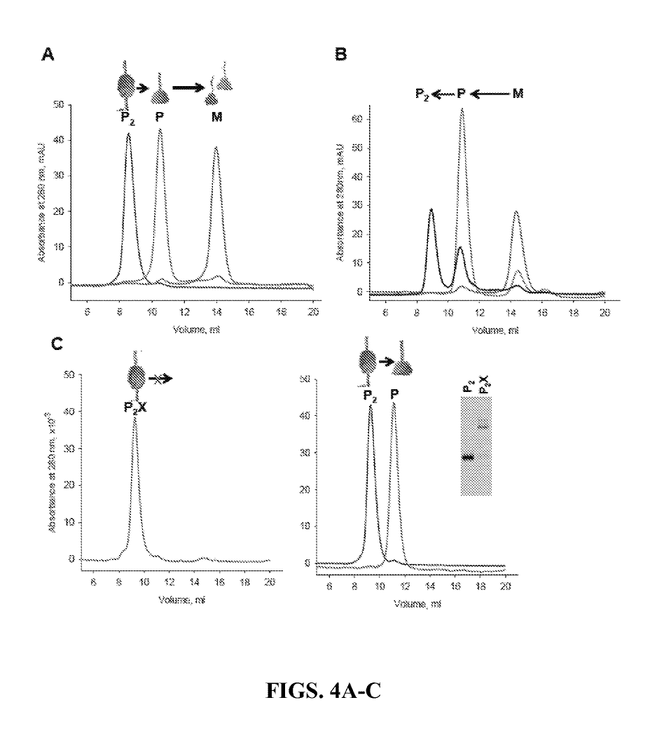

Building on the inventors' LBM hexamer assembly data, the inventors next asked whether Cl− is similarly required in assembling the observed P2 population. Considering that the stability of r-Prot samples may better reflect native collagen IV protomers than isolated LBM hexamers, the inventors viewed their recombinant system as an advanced model of scaffold assembly. Upon dialysis in TrisAc buffer, the P2 peak shifted to P (FIG. 4A), evidencing the dissociation of dimerized protomers into isolated protomers. Temperatures above the Tm of triple helix further dissociated the P population into α1 and α2 monomers (FIG. 4A), highlighting the stabilizing role of triple helical domain in collagen IV protomers. In contrast, incubation of monomers in TrisAc buffer at room temperature for 24 h induced the formation of peak P (FIG. 4B), indicating that protomer assembly relies solely on NC1 domains and does not require Cl⁻. However, a P2 peak emerged upon subsequently incubating P in the presence of Cl⁻. Hence, the NC1 domain directs protomer self-assembly independent of Cl⁻, whereas Cl⁻ triggers NC1 hexamer assembly during scaffold formation. Considering that only the extracellular space is known to possess Cl⁻ concentrations that are comparable those required here for assembly, the inventors surmised that Cl⁻ is an extracellular signal of scaffold assembly.

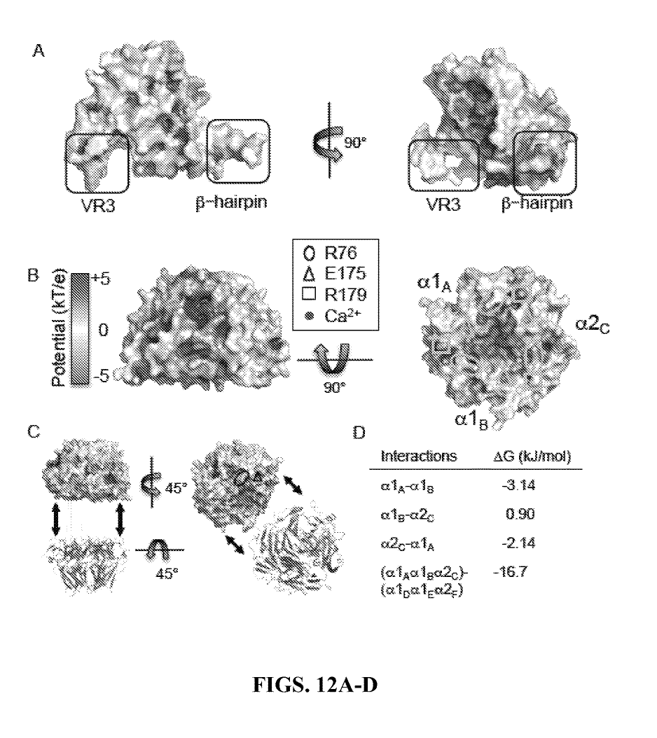

To examine how Cl− influences protomer dimerization but not protomer assembly, the inventors analyzed the binding surfaces of NC1 monomers and trimers. The inventors modeled the electrostatic potentials of α1 and α2 monomers as well as α112 trimers, finding a disparity in surface charge distribution among the three forms (FIGS. 12A-D, Table S1). Both α1 and α2 NC1 monomers have strong electronegative potential along their interior surface with both negative and positive patches on their exterior, relative to a fully formed α112 NC1 hexamer. The β-hairpin and VR3 regions, motifs essential for protomer assembly and selectivity, are mostly charge neutral in both. In contrast, the α112 protomer interface has a highly electronegative core with a discrete alternating concentric electrostatic recognition motif comprised of residues R76 and E175. To assess the potential functional impact of these differences, the inventors used nonlinear Poisson-Boltzmann calculations to estimate the impact of salt concentration on electrostatic contributions to the binding free energy (ΔG^{el}) of NC1 domains (Garcia-Garcia and Draper, 2003). The inventors found an 8-fold more favorable impact on the binding free energy for hexamer assembly over protomer assembly, suggesting that Cl⁻ functions at the level of hexamer assembly (FIG. 12D).

Cl-Dependent Conformational Switch Triggers Protomer-Protomer Assembly.

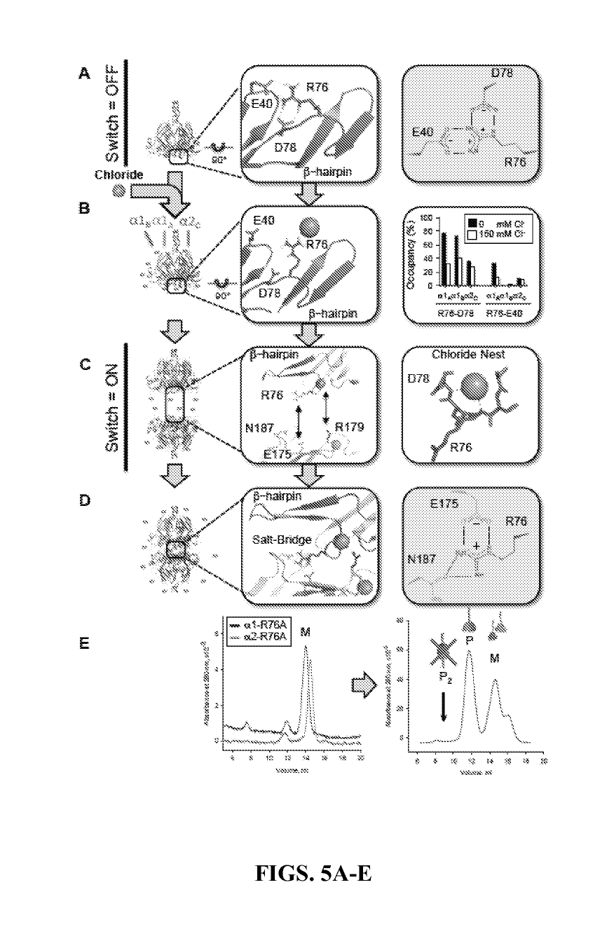

The inventors sought to understand how Cl-binding triggers hexamer assembly yet has no effect on forming protomers from monomeric chains. Using molecular modeling, they analyzed the crystallographic location of Cl− within the hexamer and noted that the ion sits in a nest formed by residues A74, S75, R76, N77, & D78 within each NC1 domain (FIG. 3A). Within this nest, Cl− coordinates the R76 and D78 backbone amide groups. Residue R76 bridges the trimer:trimer interface to form a bidentate, side-on inter-protomer interaction with E175. In addition R76 networks with N187 by hydrogen bonding in an end-on configuration (FIG. 5D), altogether creating a rare motif termed a bridging-networked salt-bridge (Donald et al., 2011). Finally, Cl⁻ mediates 6 additional electrostatic interactions at the protomer-protomer interface by directly coordinating across the protomer interface with the side chain of R179.

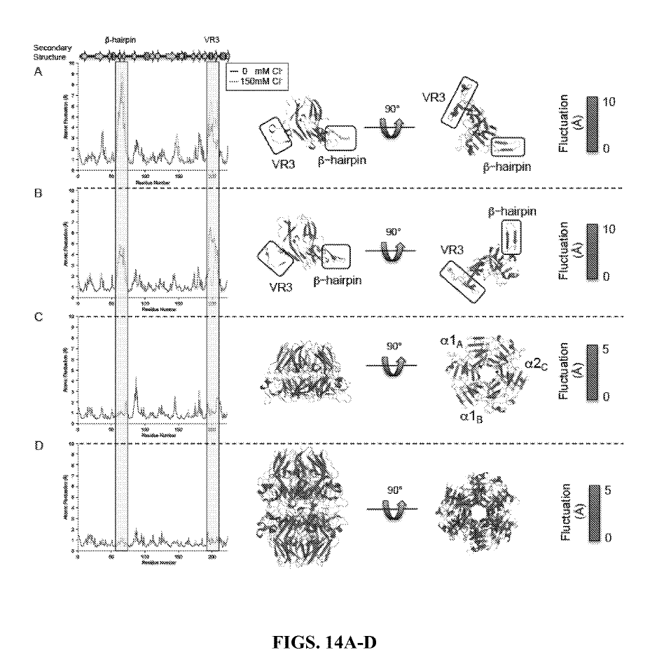

The Cl-binding nest is adjacent to the trimer-trimer interface as well as the β-hairpin motif, rather than at an α-helical termini as other nests have been described (Pal et al., 2002; Watson and Milner-White, 2002). Considering that this location may potentially influence protomer assembly, via the β-hairpin (Khoshnoodi et al., 2006b), as well as hexamer assembly, the inventors used MD simulations to model any potential influence of Cl− on the β-hairpin and better understand their assembly studies with the r-Prot. As expected, the inventors observed the β-hairpin region being highly dynamic in the monomer state yet rigid in the trimer and hexamer conformation (FIGS. 14A-D). Importantly, a Cl-induced pattern was not discernable. Concluding that Cl-binding does not have an obvious structural effect on the β-hairpin region, the inventors directed their search towards any evidence of Cl-induced conformational changes occurring at the trimer-trimer interface.

Using MD simulations, the inventors modeled residue-specific changes occurring in response to 150 mM Cl− in the bulk solvent. In the α112 trimer as well as both α1 and α2 monomers, the inventors observed that R76 forms an intra-monomer salt-bridge with D78 and to a lesser extent E40 in the absence of Cl− in the bulk solvent (FIG. 5A). In contrast, occupancy of the R76-D78 salt-bridge is reduced as much as 45% in the presence of Cl⁻ (FIG. 5B). Upon disruption of the R76-D78 interaction, the MD results predict that solvent-located Cl⁻ ions provide charge balance to the R76 side-chain through non-specific Debye-Hückel electrostatic screening, effectively preventing the intra-molecular salt bridge from reforming (FIG. 5B). Following this, since crystallographic Cl⁻ coordinates the amide backbones of R76 and D78, the inventors conclude that site-specific Cl⁻ binding within the nest restricts the available side chain conformations and repositions R76 to enable hexamer assembly (FIGS. 5C-D) via the inter-protomer, bridging-networked salt-bridge which joins the two NC1 trimers. As these inter-protomer salt bridges receive little solvent exposure within the resulting assembled hexamer, they are likely protected from additional solvent based Cl⁻ ions that might disrupt the nascent NC1 hexamer.

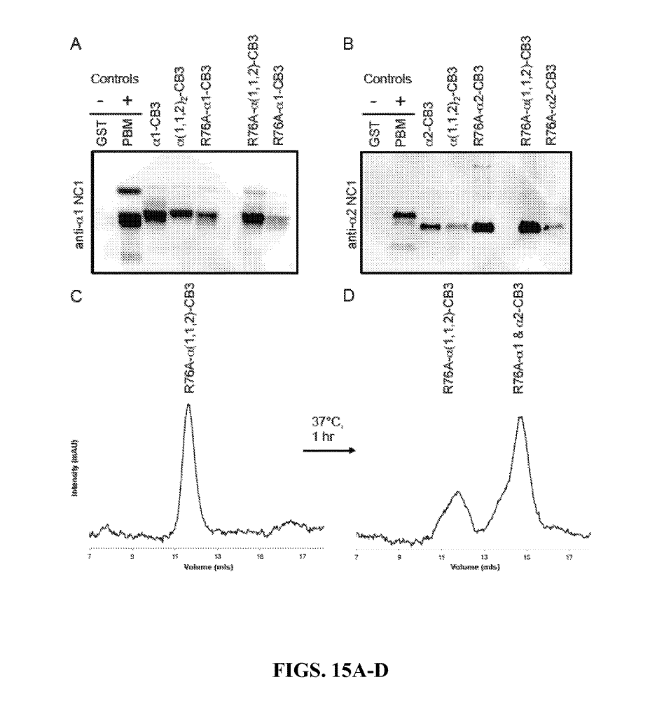

In order to test whether R76 is indeed essential for protomer-protomer assembly, the inventors generated R76A mutations of both α1 and α2 recombinant protomers and examined their ability to form P2 products. In 100 mM Cl⁻, monomers assembled into protomers, yet they did not proceed to form the P2 peak (FIG. 5E, FIGS. 15A-D). Therefore, the inventors conclude that R76 is a critical residue of the Cl-mediated mechanism during collagen IV network assembly.

Switch Residues are Defining Features of Collagen IV Scaffolds.

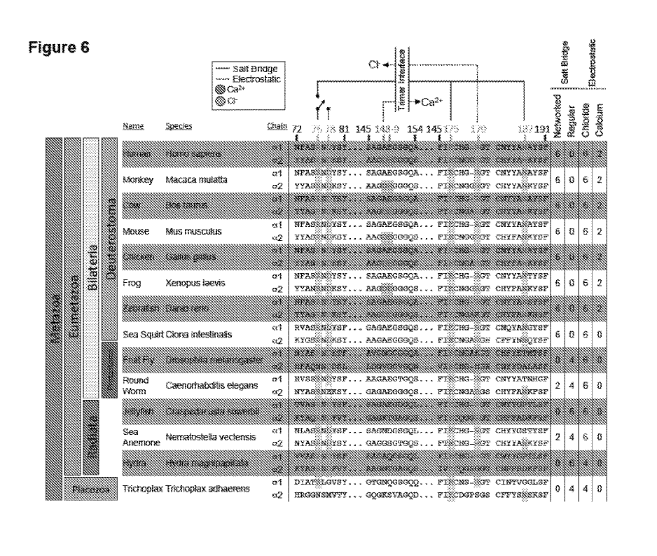

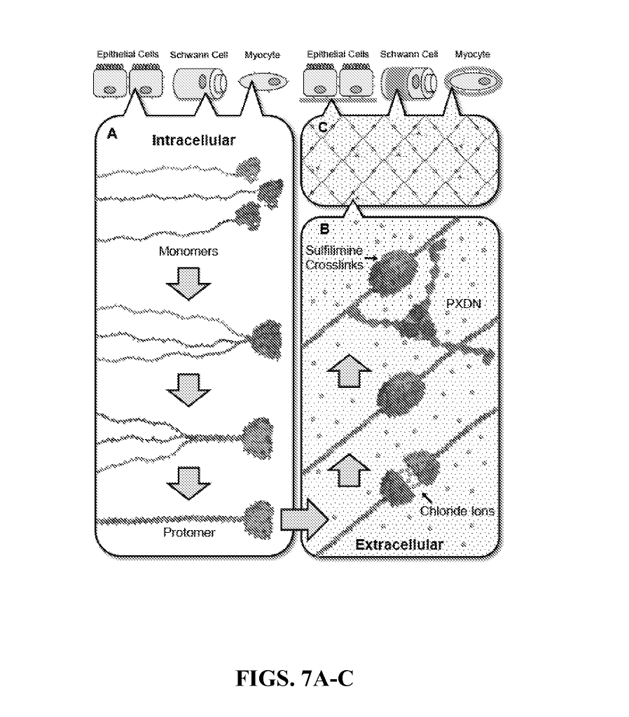

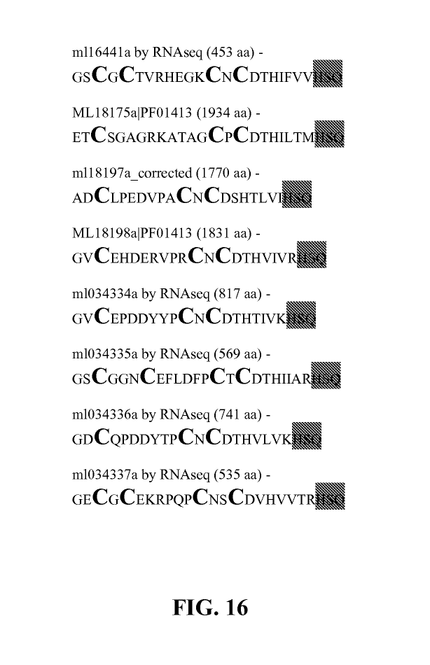

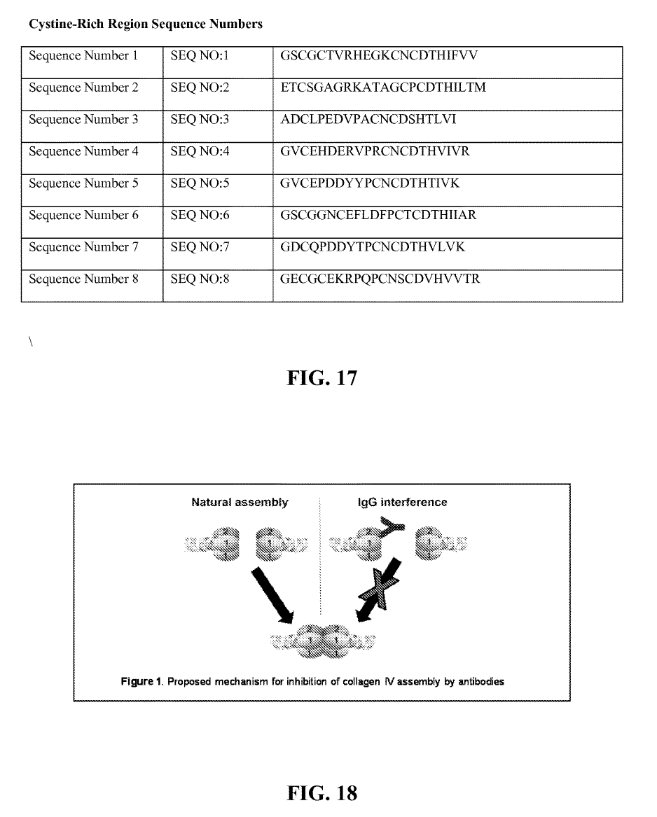

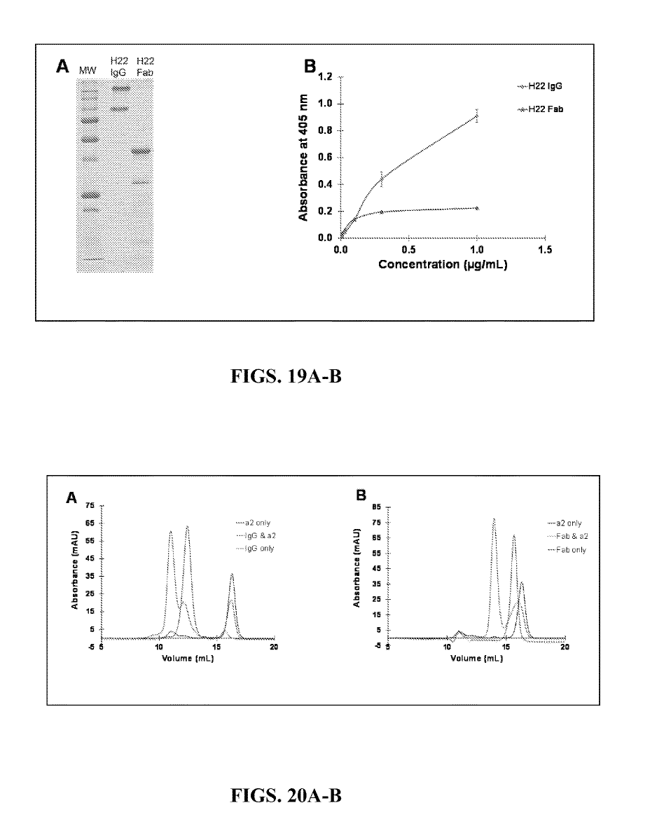

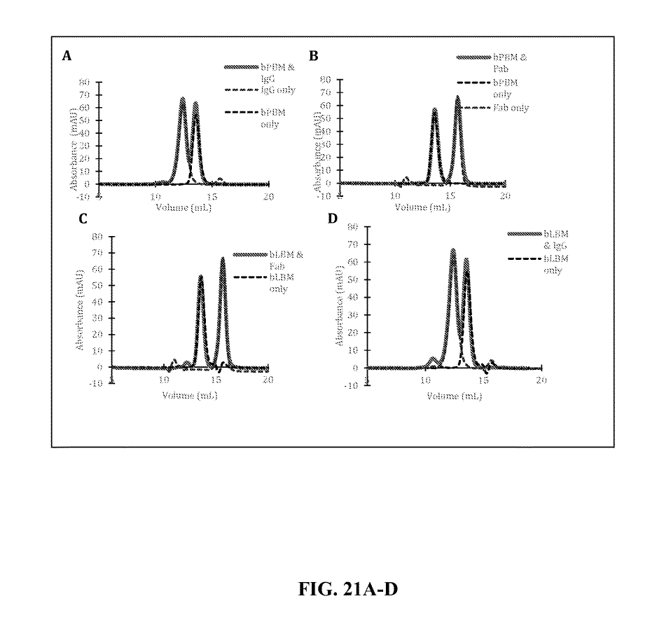

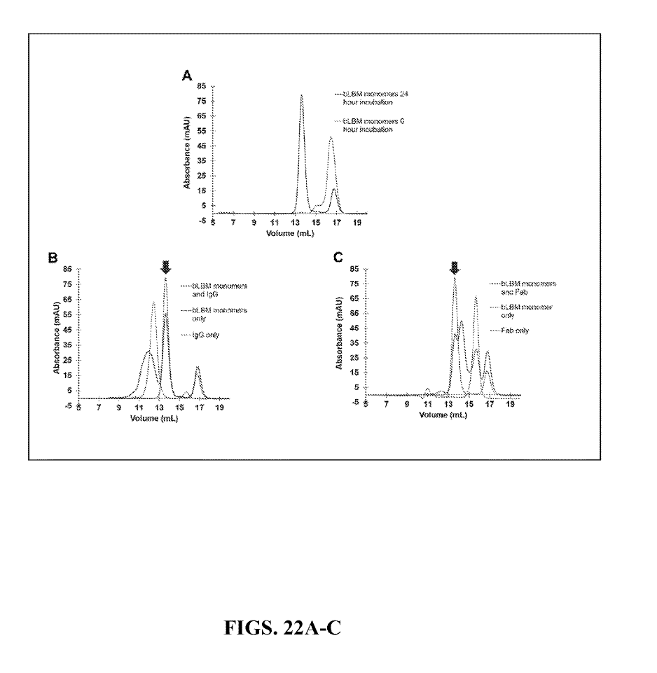

The inventors next asked if the Cl-mediated conformational switch is found throughout the Animal Kingdom, as is the sulfilimine crosslink (Fidler et al., 2014). The inventors performed a multiple sequence alignment of NC1 domains selected from organisms representing humans through Placozoa. The principal salt-bridge residue R76 and E175 are conserved in either the α1 or α2 chain through Placozoa (FIG. 6). The inventors noted that residue N187 is restricted to Deuterostoma, suggesting that R76-E175 salt bridge adopts a networked structure in this superphylum only. Residue D78 is essential for stabilizing the “off” conformation of the switch and was observed throughout Eumetazoa. R179, seen to directly interact with bound Cl⁻ in MD simulations, was found in nearly all α1 chains and most α2 chains, with Drosophila melanogaster and Zebrafish displaying potential conservative substitutions at this location. Intriguingly, Ca²⁺ binding residues D148 and E149 were exclusively present in the vertebrate α2 chain as well as the Zebrafish α4, implying an undefined function. The vertebrate α1-6 chains all displayed R76, D78, and R175 (FIG. 16). In sum, the inventors conclude that the core Cl-induced conformational switch residues are defining features collagen IV scaffolds, thereby comprising a putatively common mechanism of scaffold assembly.

Cl-Mediated Formation of the Collagen IV Network is a Prerequisite for the Final Crosslinking Step by PXDN.

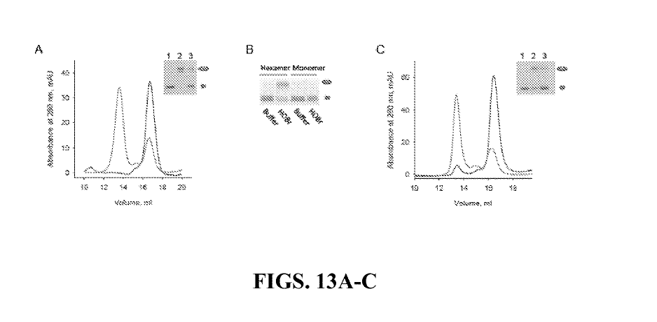

Given the extracellular localization of peroxidasin (PXDN) enzyme, which catalyzes the formation of sulfilimine crosslink (McCall et al., 2014; Bhave et al., 2012), the inventors hypothesized that their recombinant P2 population of protomer dimers would represent an appropriate substrate for peroxidasin. To test this, the inventors incubated purified P2 (FIG. 4A) with recombinant PXDN in the presence of hydrogen peroxide and Br− as co-factors. Indeed, this treatment yielded rapid crosslinking of NC1 domains as indicated by SDS-PAGE (FIG. 4C, inset). Importantly, the formation of crosslinks rendered the protomer dimers resistant to dissociation in Cl-free environment while uncrosslinked P2 remained dissociable (FIG. 4C). Similarly, PXDN-crosslinking of naturally occurring LBM hexamers, which had been reassembled from NC1 monomers, conferred resistance to dissociation (FIG. 13A). As the catalytic intermediate of PXDN-mediated crosslinking, the inventors were able to crosslink PFHR9 hexamers using hypobromous acid (HOBr) while dissociated NC1 monomers were not crosslinked by HOBr (FIG. 13B; Bhave et al., 2012). Similarly, HOBr crosslinking rendered LBM hexamers resistant to dissociation (FIG. 13C). Most strikingly, introduction of sulfilimine crosslinks rendered hexamers resistant even to strong dissociative treatment with guanidine. The inventors suggest this latter point provides direct biochemical evidence of the BM splitting and thickening the inventors have described in Br-deficient Drosophila that lack sulfilimine crosslinks (McCall et al., 2014). Together, the data indicates an extracellular pathway of scaffold assembly whereby extracellular Cl⁻ signals hexamer assembly followed by peroxidasin-catalyzed sulfilimine crosslink formation, which is critical to BM function.

Molecular Basis of Pathogenic NC1 Mutations in Alport Syndrome.

Recognizing that the R76A NC1 point mutation blocked collagen IV protomer dimerization (FIG. 5E), the inventors asked whether any NC1 mutations are known in Alport's disease, which disrupts α345 collagen IV scaffolds. Using the LOVD database (ref), the inventors cataloged 21 X-linked Alport point mutations (Table S2) in the α5 chain NC1 domain and modeled their potential structural impact. They noted that 7% of 121 families studied possess the L1649R mutation, located in the hydrophobic interior of the hexamer, and is the most common NC1 Alport mutant reported. Moreover, they found reports for an additional five cysteine point mutations that break conserved disulfide bonds. Three mutations are located along the monomer-monomer interface, two along the Ea-Eb interface. In mouse studies with α112 collagen IV, the inventors also note the recent report of an NC1 point mutation that resulted in increased levels of intracellular collagen IV (Kuo et al., 2014). For Alport's disease, the inventors propose that point mutations in the NC1 domain may disproportionally interfere with the assembly of protomers or scaffolds as a causative pathologic mechanism in some patients.

### Example—Discussion

Proper network assembly is pivotal for imparting scaffold functionality to collagen IV, evidenced by the developmental defects and lethality that result from network perturbation (Nagai et al., 2000; Matsuoka et al., 2004; Bhave et al., 2012; Pokidysheva et al., 2013; McCall et al., 2014). The process of assembly spans both sides of the plasma membrane, requiring NC1 domains to steer intracellular protomer assembly while Cl⁻ and Br⁻ are required for extracellular network assembly and crosslinking, respectively. The work presented herein illumines important steps in scaffold assembly and represents vulnerabilities that may be exploited in disease.

NC1 Activity in Protomer Assembly and Molecular Pathology.

NC1 domains self-associate through a pattern recognition process governing chain selectivity (Boutaud et al., 2000; Sundaramoorthy et al., 2002; Khoshnoodi et al., 2006b). These data shows that this interaction is critical for chain registration as well, leading to the de novo formation of active helical binding sites. Considering that the helical domain contains numerous binding sites, the inventors reason that NC1 domains may similarly influence many diverse collagen IV functions due to their role in chain selection and registration.

While genetic mutations are documented across the length of protomers, the inventors suggest that NC1-located mutations are uniquely poised to disrupt the process of assembly. Particularly, the inventors suspect that mutations within or near the pattern recognition domains may impair protomer assembly, likely preventing collagen IV secretion. Alternatively, mutations within the switch region and/or Cl-binding site may interfere NC1 hexamer formation, provided that the mutant protomer was secreted. In Alport's Syndrome, which damages α345 and/or α556 protomers (Hudson et al., 2003), some patients indeed display NC1 mutations including one case of a point mutation located adjacent to the Cl-binding nest (FIG. 14B) (Lemmink et al., 1993). Such assembly-damaging mutations may be functionally distinct from mutations within specific binding sites, with the latter potentially interfering with protomer bioactivity (Kuo et al., 2014). The recombinant strategy described herein may allow the pathologic impact of these clinical mutations to be examined in molecular detail.

Distinct Requirements for Cl⁻ and Br⁻ in Scaffold Assembly.

Halides have emerged as critical components of scaffold assembly, shown here to comprise a dual-halide mechanism where Cl⁻ and Br⁻ perform distinct and sequential functions. The inventors suspect that Cl-driven hexamer formation is important for incorporating protomers into nascent collagen IV scaffolds, occurring alongside the formation of 7S and lateral associations, in agreement with evidence that Cl⁻ enhances gelation of acid-extracted lens capsule collagen IV (Nakazato et al., 1996). Notably, the normal serum concentrations of the both ions are sufficient for the respective activities, with efficient hexamer assembly occurring at 100 mM Cl⁻, yet crosslinking apparently only requires micromolar Br− levels as found in healthy adults (McCall et al., 2014). These studies emphasize the physiologic importance of maintaining both concentrations.

Assembling “Smart” Scaffolds.

The ability of collagen IV to amalgamate signaling molecules, structural proteins, and cellular receptors implies that scaffolds are involved with coordinating the complex activities of BMs. Indeed, the three types of collagen IV protomers (α112, α345, and α556) have distinct binding partners, indicating that the overall composition and properties of BMs are strongly influenced by which protomer is expressed. In Drosophila, collagen IV scaffolds regulate BMP gradient signaling (Wang et al., 2008; Sawala, Sutcliffe, and Ashe, 2012). The inventors thus view collagen IV functioning as a “SMART” scaffold, an extracellular control center that directs the flow of mechanical and signaling information during tissue organization and development.

Covalent crosslinks seem to unite the mechanical and signaling functions of collagen IV. Formation of sulfilimine crosslinks leads to compaction of collagen IV networks (McCall et al., 2014) and greatly enhances the rigidity of NC1 hexamers (FIG. 6F,G), likely influencing the positioning of binding sites within the scaffold. Notably, sulfilimine crosslinks are not seen in H. magnipapillata yet hexamers are still observed (Fidler et al., 2014). Hydra displays a simplified tissue structure (Shimizu et al., 2008) which is apparently sufficiently supported by collagen IV scaffolds that lack sulfilimine crosslinks. The inventors therefore suggest that NC1 hexamers are basic structural pillars of collagen IV scaffolds, and that crosslinks modify scaffold functionality. As with future Alport's studies, recombinant protomers with tailored activities may allow the complexity of scaffold assembly and functionality to be elucidated in molecular detail.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
EP Application 125,023
EP Application 171,496
EP Application 173,494
EP Application 184,187
PCT Application PCT/US86/02269
PCT Application WO 86/01533
U.S. Pat. No. 4,196,265
U.S. Pat. No. 5,770,219,
U.S. Pat. No. 5,783,208
U.S. Pat. No. 6,280,766
U.S. Pat. No. 6,348,450
U.S. Pat. No. 6,555,131
Akomeah et al., Eur. J. Pharm. Sci., 21(2-3):337-45, 2004.
Baxter and Mitragotri, J. Control. Release, 106(3):361-73, 2005.
Bhave et al., Nat Chem Biol 8, 784-790, 2012.
Booji et al., AMD & Bruch's membrane review, Prog Ret Eye Res, 2010.
Borza et al., J. Biol. Chem., 280:27147, 2005.
Bramson et al., Gene Ther., 10(3):251-60, 2003.
Burnier et al. col IV & liver metastasis; dissertation ref, 2011.
Campbell and Stull, J Biol Chem 278, 12599-12600, 2003.
Case et al., J Comput Chem 26, 1668-1688, 2005.
Cornell et al., J Am Chem Soc 118, 2309-2309, 1996.
Court et al., Curr Opin Neurobiol 16, 501-507, 2006.
Daley and Yamada, Curr Opin Genet Dev 23, 408-414, 2013.
Donald et al., Proteins: Structure, Function, and Bioinformatics 79:898-915, 2011.
Emsley et al., Cell 101, 47-56, 2000.
Farrar and Carson, Biol Reprod 46:1095-1108, 1992.
Fidler et al., Proc. Natl. Acad. Sci. U.S.A., 111(1), 331-336. doi: 10.1073/pnas.1318499111.
Epub 2013 Dec. 16, 2014.
Fox et al., Cell, 129:179, 2007.
Fu et al., J. Biol. Chem. 288(11):7430-7437. doi: 10.1074/jbc.R112.444158, 2013.
Garcia-Garcia and Draper, J Mol Biol 331, 75-88, 2003.
Ghajar et al., Nat. Cell Biol. 15(7):807-817. DOI: 10.1038/ncb2767, 2013
Gotenstein et al., Development. 137:3603-3613, 2010.
Gould et al., N Engl J Med 354, 1489-1496, 2006.
Gould et al., Science, 308:1167, 2005.
Hagios et al., Philos Trans R Soc Lond B Biol Sci 353, 857-870, 1998.
Hornak et al., Proteins: Structure, Function, and Bioinformatics 65, 712-725, 2006.
Hudson et al., N. Engl. J. Med., 348:2543, 2003.
Hynes, Cell, 110:673, 2002.
Hynes, Journal of cell biology 196, 671-679, 2012.
Hynes, Science 326, 1216-1219, 2009.
Kern et al., Eur J Biochem 215, 151-159, 1993.
Khan et al., Am. J. Hum. Genet., 2011
Khoshnoodi et al., J Biol Chem 281, 38117-38121, 2006a.
Khoshnoodi et al., J Biol Chem 281, 6058-6069, 2006b.
Khoshnoodi et al., Microsc. Res. Tech., 71:357, 2008.
Kueng et al., Anal Biochem 182, 16-19, 1989.
Lochter and Bissell, Cell. 157: 1380-1392, 2014
Lu et al., J Cell Biol 196, 395-406, 2012.
Magis et al., J Am Chem Soc 128, 16190-16205, 2006.
Matsuoka et al., Mol. Biol. Cell, 15: 4467-4475, 2004.
McCall et al., Cell 157, 1380-1392, 2014.
Moser et al., Science, 324:895, 2009.
Nagai et al., J. Cell. Biol. 150(6): 1499-1505, 2000.
Nakazato et al., J. Biochem. 120(5), 889-894, 1996.
Pal et al., Angew Chem Int Edit 41, 4663-4665, 2002.
Pettersen et al., J Comput Chem 25, 1605-1612, 2004.
Ray et al., J Mol Graph Model 23, 347-354, 2005.
Sanes, J Biol Chem 278, 12601-12604, 2003.
Sawala et al., Proc. Nat. Acad. Sci. U.S.A 109:11222-11227, 2012
Sillat, et al., J Cell Mol Med 16, 1485-1495, 2012.
Song and Ott, Trends Mol Med 17, 424-432, 2011.
Strejan et al., Prog Clin Biol Res., 146:429-34, 1984.
Sundaramoorthy et al., J Biol Chem 277, 31142-31153, 2002.
Than et al., Biological Chemistry, 386:759, 2005.
Than et al., Proc. Natl. Acad. Sci. USA, 99:6607, 2002.
Tuqan et al., Lasers Med. Sci., 20(2):80-6, 2005.
Van Leeuwen and Sangster, Crit. Rev. Toxicol., 18:189, 1987
Vanacore et al., J. Biol. Chem., 279:44723, 2004.
Vanacore et al., J. Biol. Chem., 283:22737, 2008.
Vanacore et al., Science, 325:1230, 2009
Vracko et al., Am J Pathol 77, 314-346, 1974.
Wallace et al., Protein Eng 8, 127-134, 1995.
Wang et al., Nature. 455:72-77, 2008
Watson and Milner-White, J Mol Biol 315, 171-182, 2002.
Wewer et al., Lab Invest 53, 624-633, 1985.
Yurchenco and Furthmayr, Biochemistry, 23:1839, 1984.
Yurchenco, Cold Spring Harb Perspect Biol 3, 2011.

What is claimed:

1. A composition comprising three recombinant proteins, formulated in a pharmaceutically-acceptable carrier containing less than 30 mM halide ions, that assemble into a heterotrimeric complex with similarity to protomeric collagen IV, wherein:
each recombinant protein contains a C-terminal NC1 domain and a collagenous domain;
each recombinant protein in the heterotrimeric complex is independently expressed in a mammalian cell line;
the heterotrimeric complex is assembled at a temperature below 37° C. and in a solution containing less than 30 mM halide concentration; and
the heterotrimeric complex is capable of binding another heterotrimeric NC1-containing complex via the NC1 domain upon entering a solution with halide concentration above at least 30 mM.

2. The composition of claim 1, wherein the NC1 domains do not contain:
(i) an arginine residue at position 76,
(ii) a valine residue at position 27,
(iii) a leucine residue at position 29, or
(iv) an isoleucine residue at position 39
wherein said residues are numbered from the N-terminus of each NC1 domain.

3. The composition of claim 1, where at least two of the recombinant proteins comprise a cysteine-rich sequence located between the NC1 and collagenase domains, thereby inducing inter-chain disulfide crosslinks that structurally reinforce the heterotrimeric complex.

4. The composition of claim 3, where the cysteine-rich sequence is selected from SEQ ID NOS: 1-8.

5. The composition of claim 1, where the halide is chloride.

6. A method of treating a patient with Alport's Disease comprising administering to said patient an effective amount of the composition of claim 1.

7. The method of claim 6, wherein administering comprises injection into the bloodstream of the patient.

8. A method of treating a patient having or at risk of Goodpasture's Disease comprising removing collagen IV-associated autoantibodies from the bloodstream of the patient using the composition of claim 1.

9. The method of claim 8, wherein the composition is immobilized on a surface, and said the bloodstream of said patient is contact with said surface.

10. A method of treating a patient having or at risk of Goodpasture's Disease comprising administering a composition effective amount of claim 1 to the patient.

11. The method of claim 10, where the composition is injected into the bloodstream of the patient.

12. A method of treating or preventing hemorrhagic stroke in a patient comprising administering to said patient an effective amount of the composition of claim 1.

13. The method of claim 12, wherein the composition is injected into the bloodstream of the patient.

14. A method of treating a collagen IV-related disease in a patient comprising administering to said patient an effective amount of an antibody that disrupts basement membrane function by binding to collagen IV NC1 domains.

15. The method of claim 14, wherein the disease is cancer, a tumor, a metastatic tumor, or hematologic cancer.

16. The method of claim 14, where the antibody functions as an anti-angiogenesis therapy.

17. The method of claim 14, wherein the disease being treated is macular degeneration.

18. The method of claim 14, where the antibody disrupts the assembly of collagen IV NC1 hexamers.
